(19) United States
(12) Patent Application Publication
(10) Pub. No.: US 2020/0278349 A1
(43) Pub. Date: Sep. 3, 2020

(54) BIOHAZARD ANALYZER

(71) Applicant: UNIVERSITY OF UTAH RESEARCH FOUNDATION, Salt Lake City, UT (US)

(72) Inventors: Ugochukwu Chinedu Nze, Carson, CA (US); Himanshu Jayant Sant, Salt Lake City, UT (US); Bruce Kent Gale, Taylorsville, UT (US); Michael Beeman, Salt Lake CIty, UT (US); Christopher J. Lambert, Salt Lake City, UT (US)

(21) Appl. No.: 16/805,634

(22) Filed: Feb. 28, 2020

Related U.S. Application Data
(60) Provisional application No. 62/811,617, filed on Feb. 28, 2019.

(51) Int. Cl. G01N 33/553; G01N 33/543; G01N 27/12

(52) U.S. Cl. CPC G01N 33/553; G01N 27/125; G01N 33/54346

(57) ABSTRACT

Detecting a pathogen may include introducing a nonmagnetic metal into the sample where the nonmagnetic metal includes an antibody that is specific to the pathogen to form a complex of the nonmagnetic metal and the pathogen, removing the nonmagnetic metal that is not complexed with the pathogen from the sample, and detecting the presence of the nonmagnetic metal in the sample where the presence of the nonmagnetic metal indicates the presence of the pathogen.

### CROSS-REFERENCE TO RELATED APPLICATIONS

This application claims priority to and the benefit of U.S. Provisional Patent Application Ser. No. 62/811,617, filed Feb. 28, 2019 and titled “BIOHAZARD ANALYZER.” The foregoing is incorporated herein by this reference in its entirety.

### BACKGROUND

Food and water contamination contribute to many illnesses throughout the world. Due to these pathogens, using natural sources of water or food can pose health risks in underdeveloped countries as well as to military deployments and to recreationists.

### SUMMARY

The principles disclosed herein includes a method for detecting a pathogen. This method may include (i) introducing a nonmagnetic metal into the sample that is coupled to an anti-pathogen antibody specific for the pathogen and configured to form a complex of the nonmagnetic metal and the pathogen, (ii) removing free nonmagnetic metal from the sample that is not forming the complex, (iii) detecting a presence of the nonmagnetic metal in the sample, and (iv) determining a concentration of the pathogen in the sample based on the presence of the nonmagnetic metal.

In one aspect, the nonmagnetic metal includes at least one non-ferrous metal nanoshell. The nonmagnetic metal can include, for example, gold.

In one aspect, the method further includes collecting the complex of the nonmagnetic metal and the pathogen on a surface of an electrode. The complex can further include magnetic objects that are complexed with the anti-pathogen antibody. In one aspect, detecting the presence of the nonmagnetic metal in the sample includes performing voltammetry on the electrode and can additionally include comparing a signal from the voltammetry to signals from other samples known to contain the pathogen.

In one aspect, the method includes introducing the magnetic objects into the sample prior to introducing the nonmagnetic metal and can additionally include magnetically separating immunocaptured complex from a remaining portion of the sample. The immunocaptured complex can include the magnetic objects, the nonmagnetic metal, the pathogen, and the anti-pathogen antibody. In one aspect, the method can additionally include holding the magnetic objects in place within a tube with a magnet while removing non-target debris from the tube.

In one aspect, the method includes depositing a portion of the nonmagnetic metal bound to the pathogen on an electrode through an aqueous solution and drying the electrode with the deposited nonmagnetic metal bound to the pathogen. Determining the concentration of the pathogen in the sample based on the presence of the nonmagnetic metal can then include passing an electrical signal through the electrode, measuring a resulting electrical characteristic of the electrical signal, and comparing the resulting electrical characteristic to a database of electrical characteristics of known pathogens concentrations to determine the presence of a pathogen in the sample.

Embodiments of the present disclosure additionally include systems for detecting at least one pathogen in a sample. An exemplary system can include (i) a first antibody coupled to a nonmagnetic metal, (ii) a second antibody coupled to a magnetic object, wherein the first and second antibodies are configured to form a complex comprising a pathogen, the nonmagnetic metal and the magnetic object, (iii) a sample mixing chamber for receiving the sample, (iv) a magnet selectively positionable to be adjacent the sample mixing chamber, (v) an electrode in selective communication with the sample mixing chamber, the nonmagnetic metal configured to associate with the electrode, and (vi) a voltmeter in electrical communication with the electrode.

In one aspect, the first antibody and the second antibody are each specific for the pathogen.

In one aspect, the first antibody is specific for the pathogen and the second antibody is specific to the F_{c} region of the first antibody.

Embodiments of the present disclosure additionally include other methods for detecting a pathogen in a sample. Another exemplary method includes (i) binding a pathogen to a magnetic object by introducing the magnetic object into the sample, the magnetic object being complexed with a first antibody that is specific to the pathogen to form a first complex, (ii) forming a second complex including the first complex and a nonmagnetic metal by introducing the nonmagnetic metal into the sample with the first complex, the nonmagnetic metal being complexed with a second antibody specific to the pathogen, (iii) retaining the second complex in the sample with a magnet, (iv) removing a portion of the nonmagnetic metal not incorporated into the second complex, and (v) detecting the presence of the nonmagnetic metal in the sample, the presence of the nonmagnetic material indicating the presence of the pathogen.

In one aspect, the specificity of the first antibody and the second antibody are to the same epitopes of the pathogen.

In one aspect, the method further includes depositing the second complex on an electrode through an aqueous solution. The method can further include determining a concentration of the pathogen in the sample based on the presence of the nonmagnetic metal. In such instances, determining the concentration of the pathogen in the sample based on the presence of the nonmagnetic metal can include passing an electrical signal through the electrode, measuring a resulting electrical characteristic of the electrical signal, and comparing the resulting electrical characteristic to a database of electrical signals of known pathogens concentrations to determine the concentration of the pathogen in the sample.

Any of the aspects of the principles detailed above may be combined with any of the other aspect detailed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings illustrate various embodiments of the present apparatus and are a part of the specification. The illustrated embodiments are merely examples of the present apparatus and do not limit the scope thereof.

FIG. 1 depicts an example of a magnetic object complexed with an antibody attached to a pathogen in accordance with the present disclosure.

FIG. 2 depicts an example of controlling, with a magnet, a position of a pathogen bound to a magnetic object through an antibody in accordance with the present disclosure.

FIG. 3 depicts an example of a nonmagnetic metal complexed with antibodies attached to pathogens in accordance with the present disclosure.

FIG. 4 depicts an example of a magnetic object-pathogen-metal complex in a chamber in accordance with the present disclosure.

FIG. 5A depicts an example of complexes in an aqueous solution with an electrode in accordance with the present disclosure.

FIG. 5B depicts an example of complexes in an aqueous solution with an electrode where a magnet can control a position of the complexes in accordance with the present disclosure.

FIG. 6 depicts an example of a portion of an analyzing system to test for a presence of a pathogen in accordance with the present disclosure.

FIG. 7 depicts an example of a method of analyzing a sample for pathogens in accordance with the present disclosure.

FIG. 8 depicts another example of a method of analyzing a sample for pathogens in accordance with the present disclosure.

FIG. 9 depicts results of analyzing attachment of antibodies to gold nanoparticles in accordance with the present disclosure.

FIGS. 10A-C depict results of detecting gold in accordance with the present disclosure.

FIG. 11 depicts results of detecting gold nanoshells in accordance with the present disclosure.

FIGS. 12A-B depict results of detecting gold in accordance with the present disclosure.

FIG. 13 depicts results of detecting wavelength absorbance in accordance with the present disclosure.

FIGS. 14A-B depict results of bound magnetic bead accordance with the present disclosure.

FIG. 15 depicts an example of an apparatus for testing for a presence of a pathogen in accordance with the present disclosure.

FIG. 16 depicts an example of an SEM image of gold nanoshells bound to magnetic beads in a complex in accordance with the present disclosure.

FIG. 17 depicts results of detecting pathogens in accordance with the present disclosure.

FIGS. 18A-D depict an automated pump driven using a stepper motor in accordance with the present disclosure. FIG. 18A is a top view of the illustrated system; FIG. 18B is a perspective view of the illustrated system; FIG. 18C is a front view of the illustrated system; and FIG. 18D is a side view of the illustrated system.

FIGS. 19A-D depict an automated pump system using a servo driven linear actuator in accordance with the present disclosure. FIG. 19A is a perspective view of a servo motor actuated syringe pump for use with the automated pump system; FIG. 19B is a side view of the servo motor actuated syringe pump illustrated in FIG. 19A; FIG. 19C is a front view of an automated syringe pump device array; and FIG. 19D is a top view of the automated syringe p ump device array of FIG. 19C.

FIG. 20A illustrates a diagram of an antibody-based magnetic-particle-virus-gold-nanoparticle complex in accordance with the present disclosure.

FIGS. 20B-20D are graphs illustrating electrochemical detection results using the system diagrammatically illustrated in FIG. 20A in accordance with the present disclosure.

FIG. 21A illustrates a diagram of a dual-aptamer-based magnetic-particle-virus-gold-nanoparticle complex in accordance with the present disclosure.

FIGS. 21B-21D are graphs illustrating electrochemical detection results using the system diagrammatically illustrated in FIG. 21A in accordance with the present disclosure.

FIG. 22A illustrates a diagram of an immobilized-antibody-virus-aptamer-bound-gold-nanoparticle complex in accordance with the present disclosure.

FIG. 22B is a graph illustrating electrochemical detection results using the system diagrammatically illustrated in FIG. 22A in accordance with the present disclosure.

Throughout the drawings, identical reference numbers designate similar, but not necessarily identical, elements.

### DETAILED DESCRIPTION

The principles disclosed herein provide a method for testing a sample for the presence of pathogens. The term “pathogen,” as used herein, includes the corpus of parasites (or other eukaryotic organisms), bacteria, and viruses that can cause disease in humans or animals, typically via ingestion of contaminated food or water. The term “pathogen” should also be understood to include opportunistic pathogenic parasites and bacteria. In some instances, the term “pathogen” is understood to include virions or viral capsids capable of causing disease in humans or other animals, typically via ingestion of contaminated food or water.

The principles herein include a method of analyzing a biohazard and an apparatus for analyzing the biohazard. The apparatus and method may be capable of both rapid and sensitive detection of pathogens. The apparatus and method may separate target molecules from other debris in a sample. Magnetic objects that are complexed with pathogen specific antibodies may be introduced into the sample. With the pathogens bound to the magnetic objects through the antibodies, the position of the pathogens may be controlled. For example, the remainder of the concentrated sample may be diluted and washed away from a chamber holding the sample while the pathogens are retained in the chamber with a magnetic influence. In some cases, a magnet may be positioned adjacent to the chamber causing the pathogens to be secured within the chamber while the remainder of the sample is free to be removed by the movement of fluid. In some cases, the magnets may be secured against a chamber that is incorporated into a centrifuge and the pathogens may be held in place by the magnets while the centrifuge is active. In some cases, the magnet's influence allows the pathogens to move within a chamber of an active centrifuge, but the movement is limited and controlled to concentrate the pathogens even more. In those situations where no pathogens are in the sample, the magnetic objects are not bound to any pathogen.

A nonmagnetic metal that is also complexed with the antibody may be added to the sample. With the magnetic objects and the nonmagnetic metal both forming connections with the antibodies, the complexes that include the nonmagnetic metal, the pathogen, and the magnetic objects may be formed if pathogens are in the sample. A further purification procedure may be used to remove the portion of the nonmagnetic metal from the sample that is not bound to an antibody or is not part of a complex. If there are no pathogens in the sample, the nonmagnetic metal does not bind to the magnetic objects and no complexes are formed. Thus, during a purification procedure where magnetism is used to hold the magnetic objects in place, the nonmagnetic metal may be removed from the sample leaving just the magnetic objects in the sample. However, if pathogens are present in the sample, the pathogens are bound by both the magnetic objects and the nonmagnetic metal through the complexed antibodies. Thus, when a purification procedure is applied to the sample, portions of the nonmagnetic metal not bound to the pathogens are removed, leaving just that portion of the nonmagnetic metal that is bound to the pathogens.

The complexes may be added to an aqueous solution with an electrode. In some cases, the complexes are removed from the chamber to be added to the aqueous solution. But, in other cases, the aqueous solution is added to the chamber where the complexes are formed. In some embodiments, the nonmagnetic metal adheres to the surface of an electrode present within the aqueous solution. The nonmagnetic metal may have the characteristic of modifying an electrical signal passed through the electrode, and modifications in the electrical signal can be detected to determine whether the complexes are adhered to the electrode. If there are no changes to the electrical signal, a determination may be made that no complexes were in contact with the electrode and that no pathogens were in the sample. Similarly, if there are modifications to the signal or at least specific modifications to the electrical signal, then a determination may be made that complexes were in contact with the electrode and that pathogens were present in the sample.

In one example, the separation may be accomplished by concentrating to a 100 mL sample down to 4 mL using vacuum filtration (or other filtration methods). Following this, non-target debris may be removed through the introduction of magnetic objects, such as beads or other objects, that have been complexed with pathogen specific antibodies. The magnetic beads may perform immunocapture of target pathogens and are then held in place inside a centrifuge tube with the aid of a magnet. All other debris may then be removed from the sample using a pipette. In one example, to amplify the presence of single pathogens in a sample, 150 nm gold nanoshells that have been complexed with pathogen specific antibodies may be introduced into the sample. The nanoshells have a high potential for loading onto the surface of pathogens which allow even very low concentration to be detected. Addition of the gold nanoshells form magnetic bead-pathogen-gold nanoshells complexes. These complexes may then be transferred to the surface a 96 well plate with each well containing a three electrode mesoelectrochemical system. The complexes may be migrated to the surfaces of the mesoelectrode via magnet concentration or through another method. Finally, square wave voltammetry or another type of voltammetry can be on each sample to detect the presence of gold in a sample. The presence of gold may be a direct indicator of pathogenic presence because gold that is not bound to a pathogen is removed from a sample using the same magnetic separation procedure. A comparison of the electrochemical signal received from the sample of interest versus a sample that is known to contain no pathogenic material may then be made. This comparison may confirm the presence and/or absence of a pathogen in a sample.

In some cases, the apparatus and/or method may be capable of testing for multiple different types of pathogenic or nonpathogenic bacteria, protozoans, parasites, or viruses, simultaneously. Tests may be run in parallel, making the total run time for a test from sampling to report under 3 hours. The device may fully automate sampling, concentration, and detection, negating the need for highly trained personnel to run a test. Limits of detection of the device may exceed the regulatory requirement in waste water effluent for many pathogens. The device may have the ability to start a new test through a timed cycle, by operator instruction, or by a connected device such as a turbidity monitor. The device may be at strategic locations and to prevent the release of contaminated food and liquids into distribution systems. The fast response time and ultra-sensitive detection of pathogens may allow the device to be used in remote locations, cruise ships and during military deployment.

Any appropriate type of nonmagnetic metal may be used. For example, a non-exhaustive list of nonmagnetic metals that may be used include, but is not limited to, gold, silver, aluminum, copper, lead, tin, titanium, zinc, brass, bronze, other precious metals, other nonmagnetic metals, alloys thereof, mixtures thereof, or combinations thereof. In some cases, the nonmagnetic metal is incorporated into a nonmagnetic carrier that is made of a material other than metal, such as plastics, composites, other types of materials, or combinations thereof. The nonmagnetic metal may have any appropriate structure. For example, the nonmagnetic metal may be incorporated into nanoshells. However, the nonmagnetic material may be incorporated into alternative forms, such as nanorods, particles, pore structures, nanoparticles, nanotubes, nanocomplexes, beads, other types of nanostructures, or combinations thereof.

The complexes may be moved towards the surface of the electrode through any appropriate mechanism. In some cases, a magnet may be used to influence the complexes to move towards the electrode. In another example, electromigration may use a high voltage difference between two electrodes to move nonmagnetic metal from one electrode to another. In some cases, a solution is used that separates the nonmagnetic metal from the magnetic objects so that just the nonmagnetic metal or at least a higher concentration of the nonmagnetic metal is attracted to the electrode.

Voltammetry may be used with the electrode to determine the presence of the nonmagnetic metal, and therefore, the presence of pathogens in the sample. In some cases, square wave voltammetry can be used to detect the presence of the nonmagnetic metal and/or the pathogens.

Voltammetry may be performed by varying the voltage applied to the electrode and recording the electrical current at each voltage on a chart. The peaks plotted on the graph may represent characteristics of the signal that indicate the presence of the nonmagnetic metal. In some cases, the procedure may include a working electrode that contacts with the nonmagnetic metal, and a reference electrode that has a known potential with which to gauge the potential of the working electrode. Depending on the set, additional auxiliary electrodes may be used. In some cases, the signals applied to the working electrode are square waves that sweep through a range of potentials. In some cases, the aqueous solution is removed from the electrode before measuring the signal characteristics. In some cases, the electrode is allowed to dry before measuring the signal characteristics. In yet other situations, the electrodes are actively dried by actively passing air across a surface of the electrode, increasing a temperature in a surrounding environment of the electrode, lowering a humidity in the surrounding environment of the electrode, or combinations thereof to promote drying of the electrode before measuring the characteristics of the electrical signal.

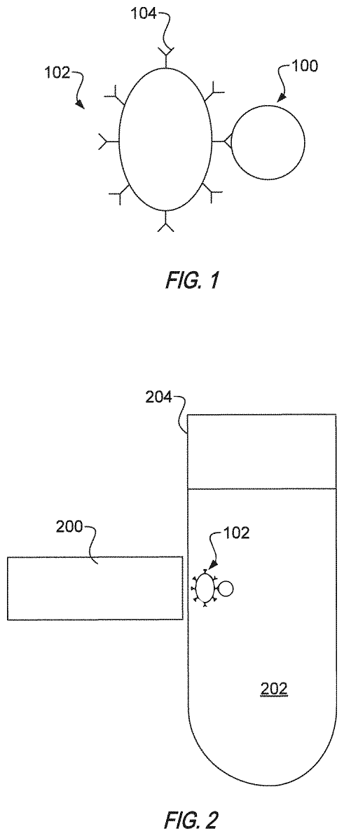

Particularly, with reference to the figures, FIG. 1 depicts an example of a pathogen 100, a magnetic object 102, and an antibody 104 connected to the magnetic object 102. In this example, the magnetic object is a magnetic bead and the antibody is attached to the magnetic bead. In this example, a single pathogen is attached to the magnetic bead, but multiple pathogens may be attached in other examples.

The magnetic bead may be complexed with an antibody that is specific to a certain type of pathogen. Any appropriate type of pathogen may be used in accordance with the principles described herein. For example, a non-exhaustive list of pathogens may include viruses, bacteria, fungi, protozoans, parasites, Cryptosporidium spp., Escherichia coli, Novovirus, Salmonella spp., other types of pathogens, or combinations thereof. Further, the sample containing the pathogen may be made from any appropriate source. For example, the source of the pathogen may be from meat, water, vegetables, fruit, plants, mushrooms, egg yolk, cheese, liquids, food, milk, juice, other types of solid foods, other types of liquid foods, medicine, ingestible materials, or combinations thereof.

FIG. 2 depicts an example of a magnet 200 influencing the position of the magnetic object 102 in an aqueous solution 202 in a sample mixing chamber 204. With the magnet 200 holding the magnetic object in place, the rest of the sample may be moved out of the chamber 204, such as by draining at least a portion of the aqueous solution, while the magnetic object, and thereof the pathogen attached to it, remain in the chamber 204 as controlled by the magnet 200.

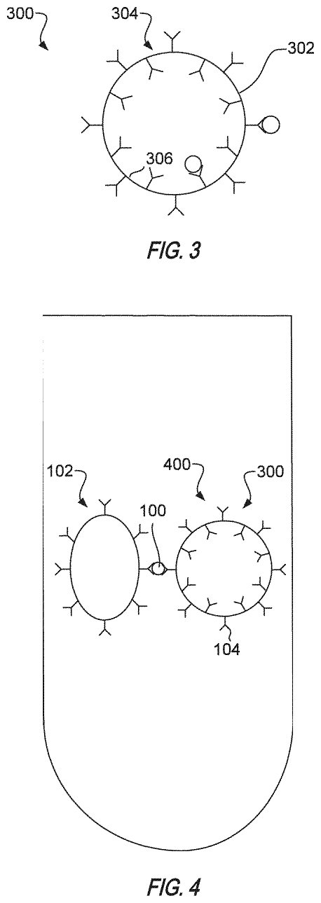

FIG. 3 depicts an example a nonmagnetic metal 300. In this example, the nonmagnetic metal 300 includes a structure of a nanoshell 302. The nanoshell 302 may include a relatively high surface area where antibodies 104 can be attached to the outside surface 304 of the nanoshell 302 and the inside surface 306 of the nanoshell 302. While the structure of the nonmagnetic metal 300 is a nanoshell 302, the nonmagnetic metal 300 may include any appropriate structure such as a porous structure, a particle structure, a nanoparticle structure, a nanotube structure, a nanorod structure, a bead, another type of structure, or combinations thereof.

FIG. 4 depicts an example of a complex 400 of nonmagnetic metal-pathogen-magnetic objects. In this example, the pathogen 100 binds to antibodies 104 of the magnetic object 102 and the nonmagnetic metal 300. These complexes 400 may be formed by allowing the magnetic objects 102 bind with the pathogens 100 in a solution and then introducing the nonmagnetic metal 300 into the solution or another solution with the pathogens 100 and the magnetic objects 102 already joined. In other examples, the nonmagnetic metal 300 and the magnetic objects 102 may bind to the pathogens 100 at the same time. In yet other examples, the nonmagnetic metal 300 may bind with the pathogens 100 before allowing the magnetic objects 102 to bind to the pathogens 100. In some examples, where the size between the nonmagnetic metal 300 and the magnetic objects 102 is significant, a higher binding rate may be achieved by binding the smaller of the two with the antibodies first.

In some cases, different size magnetic objects and/or different size metal structures may be used to increase binding for specific types of pathogens. In some situations, the size of the magnetic objects and/or metal structures may result in a high binding rate.

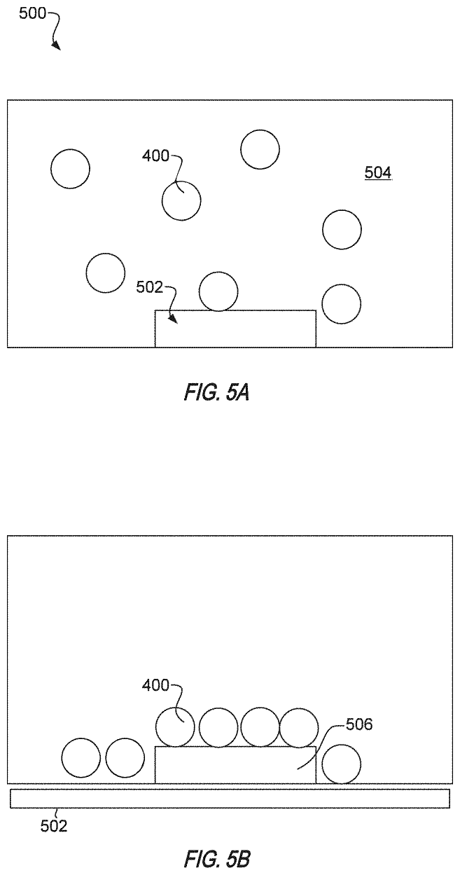

FIG. 5A depicts an example of a chamber 500 with an aqueous solution 402 and a working electrode 502 disposed within the aqueous solution 504. In this example, the complexes 400 of nonmagnetic metal-pathogen-magnetic objects are diffused throughout the aqueous solution 504. However, with a high enough concentration, the complexes may come into contact with the working electrode 502 and cause the electrical characteristics of the electrical signal to change at various voltages.

FIG. 5B depicts an example where a magnet 506 is used to direct the complexes 400 towards the electrode. In other examples, the difference in electrical potentials between a reference electrode and the working electrode 502 can be used to migrate the complexes 400 towards to the working electrode 502. While not shown in FIGS. 5A and 5B, a reference electrode may be incorporated into the chamber and used with the working electrode to perform the voltammetry.

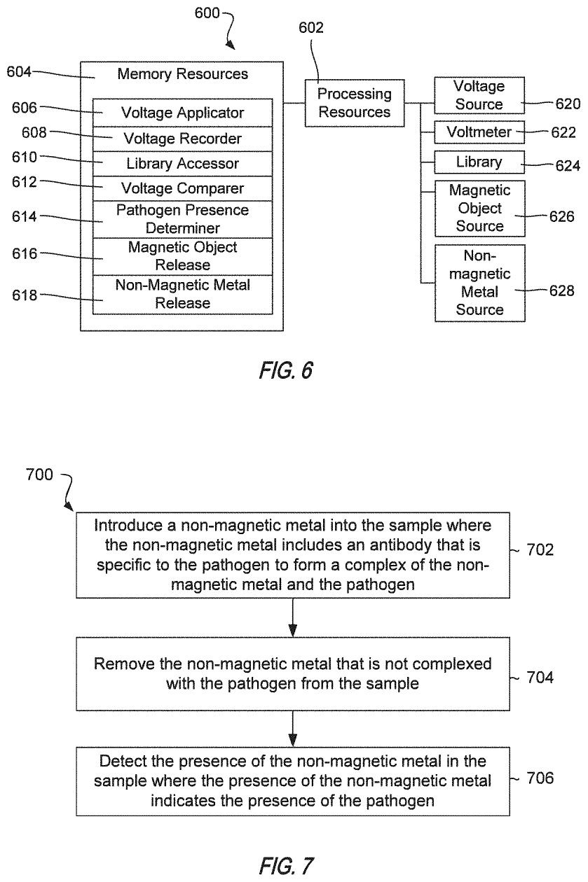

FIG. 6 illustrates a perspective view of an example of an analyzing system 600 in accordance with the present disclosure. The analyzing system 600 may include a combination of hardware and computer executable instructions for executing the functions of the analyzing system 600. In this example, the analyzing system 600 includes processing resources 602 that are in communication with memory resources 604. Processing resources 602 include at least one processor and other resources used to process the computer executable instructions. The memory resources 604 represent generally any memory capable of storing data such as computer executable instructions or data structures used by the analyzing system 600. The computer executable instructions and data structures shown stored in the memory resources 604 include a voltage applicator 606, a voltage recorder 608, a library accessor 610, a voltage comparer 612, a pathogen presence determiner 614, a magnetic object release 616, and a nonmagnetic metal release 618.

The processing resources 602 may be in communication with a voltage source 620, a voltmeter 622, a library 624, a magnetic object source 626, and a nonmagnetic metal source 628, or combinations thereof. Each of the voltage source 620, a voltmeter 622, a library 624, a magnetic object source 626, and a nonmagnetic metal source 628, the memory resources 604, and the processing resource 602 may be incorporated into a single device. In other examples, at least some of these components may be incorporated into two or more devices. In yet other examples, at least some of the code in the memory resources is located in a device with these components. But, in other examples, at least some of the memory may be accessible from a remote location, such as a cloud source.

In examples where at least some of the processing resources 602, memory resources 604, and the other components are not embodied in a single device, the processing resources 602, memory resources 604, and/or components of the mobile device may communicate over any appropriate network and/or protocol through the communications interface. In some examples, the communications interface includes a transceiver for wired and/or wireless communications. For example, these devices may be capable of communicating using the ZigBee protocol, Z-Wave protocol, Bluetooth protocol, Wi-Fi protocol, Global System for Mobile Communications (GSM) standard, another standard or combinations thereof. In other examples, the user can directly input some information into the analyzing system 600 through a digital input/output mechanism, a mechanical input/output mechanism, another type of mechanism or combinations thereof.

The memory resources 604 include a computer readable storage medium that contains computer readable program code to cause tasks to be executed by the processing resources 602. The computer readable storage medium may be a tangible and/or non-transitory storage medium. The computer readable storage medium may be any appropriate storage medium that is not a transmission storage medium. A non-exhaustive list of computer readable storage medium types includes non-volatile memory, volatile memory, random access memory, write only memory, flash memory, electrically erasable program read only memory, magnetic based memory, other types of memory or combinations thereof.

The voltage applicator 606 represents computer executable instructions that, when executed, cause the processing resources 602 to apply varying voltages from the voltage source 620 to an electrode in an apparatus for detecting the presence of pathogens. The voltage recorder 608 represents computer executable instructions that, when executed, cause the processing resources 602 to use the voltmeter 622 to measure the electrical characteristics of the signal applied to the electrode. The library accessor 610 represents computer executable instructions that, when executed, cause the processing resources 602 to access the library of previously recorded electrical characteristics of samples that have included known pathogens. The voltage comparer 612 represents computer executable instructions that, when executed, cause the processing resources 602 to compare the electrical characteristics of the current sample with those of measurements of those samples that had known pathogens. The voltage comparer 612 may identify the differences between the measurements. The pathogen presence determiner 614 represents computer executable instructions that, when executed, cause the processing resources 602 to determine whether the sample includes a pathogen. The pathogen presence determiner 614 may make the determination by judging how different the signal measurements are between the present sample and the sample with the known pathogens.

The magnetic object release 616 represents computer executable instructions that, when executed, cause the processing resources 602 to release an appropriate number of magnetic objects into a sample mixing chamber when a sample is being prepare for testing. The nonmagnetic metal release 618 represents computer executable instructions that, when executed, cause the processing resources 602 to release an appropriate amount of magnetic nonmagnetic metal into a sample mixing chamber when a sample is being prepare for testing. In some cases, the magnetic object release 616 and the nonmagnetic metal release 618 may be controlled by an automated syringe or multiple automated syringes.

In some cases, a syringe pump may be used to deliver microliter doses of magnetic beads (or other types of magnetic objects) and/or gold nanoparticles (or other structures of nonmagnetic metal) to the mixing chamber. An exemplary pump constructed for an experiment used a stepper motor controlled by an Arduino Nano micro controller along with a Pololu motor driver (Model: md09b). The Arduino Nano micro controller can be purchased from https://www.arduino.cc/ (last visited Oct. 9, 2018), and the Pololu motor driver can be purchased from the Pololu Corporation, which has a place of business at 920 Pilot Rd., Las Vegas, Nev. 98119, U.S.A. The body of the pump was 3D printed using polylactic acid, which is resistant to ethanol cleaning agents. The device may be controlled by USB serial connection or by the Arduino Mega connection. Calculations predict the syringe pump has a resolution of 0.4 μL/step using a 1 mL syringe.

Further, the memory resources 604 may be part of an installation package. In response to installing the installation package, the computer executable instructions of the memory resources 604 may be downloaded from the installation package's source, such as a portable medium, a server, a remote network location, another location or combinations thereof.

In some examples, the processing resources 602 and the memory resources 604 are located within a mobile device, an external device, networked device, a remote device, another type of device, or combinations thereof. The memory resources 604 may be part of any of these device's main memory, caches, registers, non-volatile memory, or elsewhere in their memory hierarchy. In some cases, the memory resources 604 may be in communication with the processing resources 602 over a network. Further, data structures, such as libraries or databases containing user and/or workout information, may be accessed from a remote location over a network connection while the computer executable instructions are located locally.

FIG. 7 illustrates a block diagram of an example of a method 700 of detecting the presence of a pathogen in a sample in accordance with the present disclosure. In this example, the method 700 includes introducing 702 a nonmagnetic metal into the sample where the nonmagnetic metal includes an antibody that is specific to the pathogen to form a complex of the nonmagnetic metal and the pathogen, removing 704 the nonmagnetic metal that is not complexed with the pathogen from the sample, and detecting 706 the presence of the nonmagnetic metal in the sample where the presence of the nonmagnetic metal indicates the presence of the pathogen.

At block 702, a nonmagnetic metal is introduced into a sample where the nonmagnetic metal is complexed with an antibody that is specific to a pathogen. In some cases, the nonmagnetic metal and the pathogen form a complex. In some cases, the complex includes other components, such as a magnetic object. In other cases, however, the nonmagnetic metal and the pathogen form a complex without the magnetic objects.

At block 704, the nonmagnetic metal that is not complexed with the pathogen from the sample is removed. In those examples where the complexes include a magnetic object, the magnetic objects may be used to suspend the complexes in place while other portions of the sample are removed. In those examples where no magnetic object is incorporated into the complexes, other means of removing portions of the sample with the metal that is not bound to the pathogens can be used.

At block 706, the presence of the nonmagnetic metal in the sample may be detected. The presence of the nonmagnetic metal may indicate the presence of a pathogen in the sample. The metal may be detected by introducing the complexes into a solution with an electrode that is a part of a testing circuit. The testing circuit may pass a series of voltages across the electrode and measure the corresponding electrical signals. In those cases where the electrical signal is different than would otherwise be expected based on a control group, the sample may be determined to include the pathogen due to the complexes coming into electrical contact with the electrode.

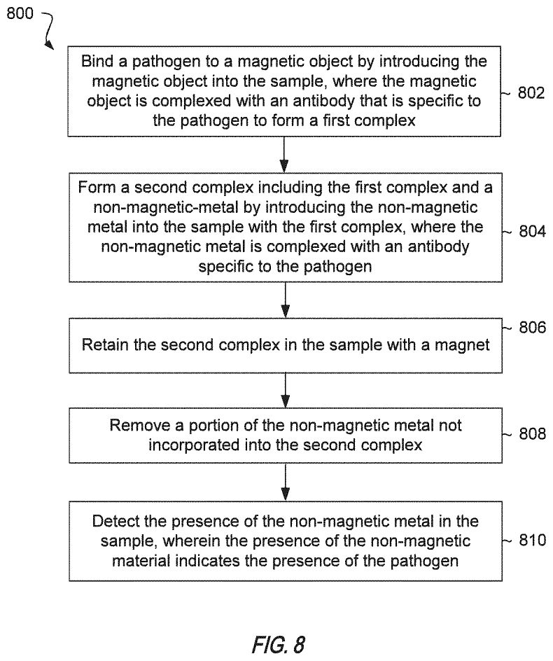

FIG. 8 illustrates a block diagram of an example of a method 800 of detecting the presence of a pathogen in a sample in accordance with the present disclosure. In this example, the method 800 includes binding 802 a pathogen to a magnetic object by introducing the magnetic object into the sample where the magnetic object is complexed with an antibody that is specific to the pathogen to form a first complex, forming 804 a second complex including the first complex and a nonmagnetic-metal by introducing the nonmagnetic metal into the sample with the first complex where the nonmagnetic metal is complexed with an antibody specific to the pathogen, retaining 806 the second complex in the sample with a magnet, removing 808 a portion of the nonmagnetic metal not incorporated into the second complex, and detecting 810 the presence of the nonmagnetic metal in the sample where the presence of the nonmagnetic material indicates the presence of the pathogen.

At block 802, the pathogens are bound to the magnetic objects by introducing the magnetic objects into the sample. The magnetic objects may be complexed with an antibody that is specific to the pathogen. The magnetic objects may include magnetic beads. In those examples where no pathogens that are specific to the antibody are in the sample, the magnetic objects may not bound with any pathogens.

At block 804, the second complexes may be formed by introducing a nonmagnetic metal into the sample. In this example, the pathogen may bind to the antibodies of the magnetic object and the antibodies of the metal. In those examples where there are no pathogens that are specific to the antibodies on the magnetic objects or on the metal, no second complexes may be formed. Rather, the metal and the magnetic objects may remain independent and unbounded to one another.

At block 806, the second complexes are retained with a magnet. This may be accomplished by positioning a magnet adjacent to the chamber containing the solution. The magnet may retain the magnetic objects while allowing the nonmagnetic metal to move freely.

At block 808, the portion of the nonmagnetic metal not incorporated into the second complex is removed. With the nonmagnetic metal is free to move despite the presence of the magnetic field, unbound nonmagnetic metal in the sample may be removed by pouring a portion of the aqueous solution out of the chamber, by draining the aqueous solution out of the chamber, by physically removing the nonmagnetic metal with a pipette, by another removal mechanism, or combinations thereof. In other examples, where there is no pathogen that is specific to the antibodies attached to the magnetic objects or the metal, the nonmagnetic metal may be removed with the sample while the pathogen-less magnetic objects are held in place.

At block 810, the presence of the nonmagnetic metal in the sample is detected. The presence of the nonmagnetic metal may indicate the presence of the pathogen. The second complexes may be deposited on an electrode. In those examples where there is no pathogen specific to the antibodies in the sample, the magnetic objects may be passed through the aqueous solution to the electrodes, but no metal would be transferred to the electrodes. An electrical current may be passed through the electrode. In some cases, multiple signals are passed through the electrode with different electrical characteristics. In some cases, the different electrical characteristics may include a voltage difference. In some cases, a voltammetry procedure is used. For example, square wave voltammetry may be applied to the electrode.

Various experiments have been conducted based on the principles described herein. The following selected experiments are presented as illustrative examples:

### EXAMPLE 1

### Preparation of Salmonella and Attachment to Magnetic Dynabeads™

In this example, freeze dried Salmonella enterica serovar typhimurium (referenced herein as “S. typhimurium” or “Salmonella”) were procured from America Type Culture Collection (ATCC 53648™). S. typhimurium were rehydrated in 5 mL of Difco™ Nutrient Broth (ref 23400). The sample was centrifuged at 1163 grams for 15 minutes to form a pellet, and the supernatant was removed. The pellet was resuspended in 3 mL of broth. Two 1-millileter aliquots of the second-generation S. typhimurium and 10% volume/volume glycerol were placed in a cryogenic freezer for future use. 100 μL of S. typhimurium were plated on Difco™ agar plates and incubated for 24 hours to confirm the viability of the S. typhimurium. Difco™ products can be purchased through FisherScientic, which has a place of business in 168 Third Avenue Waltham, Mass., U.S.A.

### EXAMPLE 2

### Attachment to Magnetic Dynabeads™

Attachment of Salmonella to the anti-Salmonella magnetic Dynabeads™ was tested by plating the constituents before and after attachment. Flow cytometry was also used to analyze the attachment of Salmonella. Salmonella samples with an OD₆₀₀ reading of near 0.1, correlating to a sample having a density of 50×10⁶ colony forming units per mL (CFU/mL), was diluted five times in a 10-fold dilution series (50×10⁵, 50×10⁴, 50×10³, 50×10², 500 CFU/mL). Diluted samples on the scale of 50×10² and 500 CFU/mL were plated because using less dilute pure Salmonella samples resulted in plates with too many CFUs to count. Samples were plated before and after attachment by spreading 50 μL of sample on Difco™ agar plates and incubating at 37° C. for 24 hours. The Dynabeads™ may also be purchased through Thermo Fisher Scientific.

### EXAMPLE 3

### Attachment of E. coli to Gold Nanoparticle/Gold Nanoshell

A two-bead system was used in this example as the method of pathogen detection. In this example, the first bead captures and isolates bacteria from all other debris that may be initially present in a given sample. The second bead is a detection bead and contains the target that may be sensed during electrochemical detection. Both beads are complexed to an anti-bacterial antibody to ensure binding of the bead to the bacterium of interests' antigen.

Before a full end-to-end detection of bacteria can be conducted using gold particles and/or gold nanoshells, the complexes of the anti-bacterial antibody to the secondary bead is confirmed. This can be achieved by measuring the absorbance spectrum of the gold nanoparticle before and after the complex formation protocol is performed. Gold nanoparticles of varying sizes have very well studied absorbance peaks, the wavelengths at which the gold nanoparticles absorb the most energy from light. However, the introduction of a complexed antibody on the surface of the gold nanoparticle causes a 5-6 nm redshift of the absorbance spectra and a slight broadening of the absorbance peak. This redshift can be used as an indicator of antibody attachment onto the gold nanoparticle and/or gold nanoshells.

In this example, four samples, hereafter referred to as samples A, B, C, and D, were created. Sample A contained 60 μL of a 1× Phosphate Buffered Saline (PBS) and was designated as a negative control. Sample B contained a mixture of 50 μL of 1× phosphate buffered saline and 10 μL of a biotinylated anti-E. coli antibody kept at a concentration of 4-5 mg/μL. Sample C contained a mixture of 50 μL of a 40 nm streptavidin coated gold nanoparticle solution and 10 μL of a biotinylated anti-E. coli antibody kept at a concentration of 4-5 mg/μL. Finally, Sample D contained a mixture of 50 μL of a 40 nm streptavidin coated gold nanoparticle solution and 10 μL of 1×PBS. All samples were allowed to mix end over end for 30 minutes to ensure proper mixing. After 30 minutes, samples C and D were centrifuged for 10 minutes at 3.6k RCF. This centrifugation resulted in the formation of a gold nanoparticle pellet. Following centrifugation, the supernatant contain unbound antibody and other debris were removed from sample C and D. All samples were then diluted to a final volume of 1.250 mL. An absorption spectrum for each sample was then collected using ultraviolet-visible spectroscopy or ultraviolet-visible spectrophotometry.

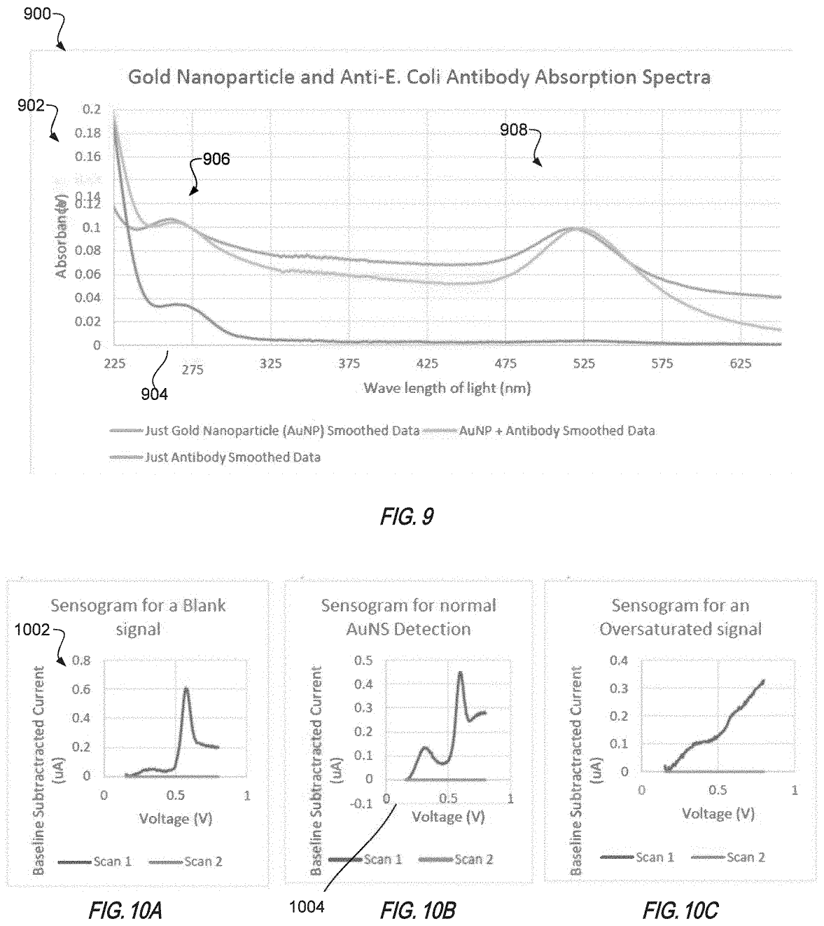

The results of this example are depicted in the chart 900 of FIG. 9. The y-axis 902 schematically represents an absorbance, and the x-axis 904 represents a wavelength of light. The absorbance spectra for each sample was measured and recorded. Sample A, the blank sample, was found to have no absorbance at any measured wavelength, indicating no protein contaminants were present in this sample. Thus, Sample A is not depicted in chart 900. Sample B displayed an absorbance peak 906 at roughly 270 nm, corresponding to the absorbance peak of just this antibody. The 40 nm gold nanoparticles that did not receive an antibody treatment displayed two absorbance peaks. The first absorbance peak 906 at 270 nm corresponded with a protein peak due to the streptavidin coating on the surface of the particle. The second peak 908 at 517 nm corresponded with the peak of just gold itself. The gold nanoparticle that did receive the antibody treatment also displayed two peaks. The first peak 906 corresponding to the presence of streptavidin was considerably blue shifted in this case. Additionally, the gold peak 908 experienced a red shift of 6 nm, occurring at 523 nm. This redshift is believed to have confirmed that the current antibody binding protocol is satisfactory for use with gold particles and/or gold nanoshells.

### EXAMPLE 4

### Detection of Gold Metal in Salmonella Sample

In this example, a presence/absence test was conducted using anti-Salmonella complexed 40 nm gold nanoparticle and 150 nm gold nanoshells. Anti-Salmonella antibody complexed 40 nm gold nanoparticles and 150 nm gold nanoshells were created. Four samples, referred to as sample A, B, C, and D, were created. Samples A and B contained 4 mL of 1× phosphate buffered saline to be used as negative controls. Sample C and D contained 4 mL of Salmonella at 50,000 CFU/mL. Each sample was aliquoted into four 1 mL divisions. Each aliquot was also allowed to complex with 1 million magnetic microspheres coated with an anti-Salmonella antibody for 40 minutes Immunomagnetic separation (IMS) was then performed on all the aliquots two times. Following IMS, aliquots from samples A and C received 2.5 μL of gold nanoparticles. Samples B and D alternatively received 2.5 μL of gold nanoshells as their treatment.

Each aliquot was allowed to mix with the gold for 40 minutes and then three immunomagnetic separation washes were performed. All aliquots were then resuspended in 100 μL of a 0.1 M HCl Solution, after which the eluent of each sample was placed on an electrochemical sensor. Each aliquot was allowed to rest on the sensor for 10 minutes before square wave voltammetry was performed. To do this, all samples were pre-conditioned at 1.3 volts for 30 seconds to oxidize all Au⁰ into Au³⁺. Following this, reduction of Au³⁺ was conducted by sweeping the potential from 0.15 volts to 0.6 volts, with a step potential of 0.004 volts, amplitude of 0.02 volts, and frequency of 100 Hz. The electrochemical signal from all samples were then recorded.

The Electrochemical signal for each sample aliquot was measured and recorded. An average sample signal was then used to determine whether gold was able to differentiate between the presence or absence of Salmonella in a sample. The gold nanoparticles differentiated between the presence and absence of Salmonella with Salmonella positive samples resulting in 165 nanoamps of electrochemical signal and Salmonella negative samples resulting in 131 nanoamps. A summary of all the results can be found in Table 1.

TABLE 1

| | Electro | Average | Co- |
|---|---|---|---|
| | chemical- | Electro- | efficient |
| | Signal | chemical | of |
| Sample ID | Received (nA) | Signal (nA) | Variance |
| Sample A (Blank AuNP 1) | 231.25 | 130.875 | 68% |
| Sample A (Blank AuNP 2) | | | |
| Sample A (Blank AuNP 3) | 61.064 | | |
| Sample A (Blank AuNP 4) | 100.314 | | |
| Sample B (Blank AuNS 1) | 228.32 | 191.628 | 37% |
| Sample B (Blank AuNS 2) | 273.5 | | |
| Sample B (Blank AuNS 3) | 130.251 | | |
| Sample B (Blank AuNS 4) | 134.439 | | |
| Sample C (Salmonella + | 125.377 | 164.824 | 44% |
| AuNP 1) | | | |
| Sample C (Salmonella + | 248.75 | | |
| AuNP 2) | | | |
| Sample C (Salmonella + | 120.344 | | |
| AuNP 3) | | | |
| Sample C (Salmonella + | | | |
| AuNP 4) | | | |
| Sample D (Salmonella + | 229.25 | 168.439 | 29% |
| AuNS 1) | | | |
| Sample D (Salmonella + | 175.128 | | |
| AuNS 2) | | | |
| Sample D (Salmonella + | 156.25 | | |
| AuNS 3) | | | |
| Sample D (Salmonella + | 113.128 | | |
| AuNS 4) | | | |

### EXAMPLE 5

### Evaluation of Gold Nanoparticles Verses Gold Nanoshells

In this example, samples A, B, and C were created. Sample A contained 400 μL of a 0.1 M HCl to be used as negative controls. Sample B contained a mixture of 10 μL of 40 nm gold nanoparticles and 390 μL of 0.1 M HCl. Sample C contained a mixture of 10 μL of 150 nm gold nanoshells and 390 μL of 0.1 M HCl. All samples were aliquoted into four 100 μL samples and square wave voltammetry was run on them using methods described previously in this report. At high concentrations, these results indicate that the gold nanoshells provide more of an electrochemical signal than gold nanoparticles. Results of this example can be found summarized in Table 2.

TABLE 2

| | Electro- | Average | |
|---|---|---|---|
| | chemical | Electro- | |
| | Signal | chemical | Coefficient |
| Sample ID | Received (nA) | Signal (nA) | of Variance |
| Sample A (Blank 1) | 101.189 | 95.183 | 8% |
| Sample A (Blank 2) | 92.689 | | |
| Sample A (Blank 3) | 85.283 | | |
| Sample A (Blank 4) | 101.969 | | |
| Sample B (AuNP 1) | 117.627 | 121.690 | 6% |
| Sample B (AuNP 2) | N/A | | |
| Sample B (AuNP 3) | 118.02 | | |
| Sample B (AuNP 4) | 129.44 | | |
| Sample C (AuNS 1) | 140.314 | 147.814 | 15% |
| Sample C (AuNS 2) | 130.252 | | |
| Sample C (AuNS 3) | 172.878 | | |
| Sample C (AuNS 4) | N/A | | |

### EXAMPLE 6

### Evaluation of Gold Nanoshell Saturation Point

This experiment was conducted to investigate the effects of concentration levels of gold nanoshells at which the electrochemical sensor becomes saturated. In some testing configurations, when a concentration level is greater than a saturation limit, the biosensor may have difficulty differentiating between samples. An illustration of this can be seen in FIGS. 10A-C. In these figures, the y-axis 1002 represent electrical current, and the x-axis 1004 represents voltage. In this example, the detection of Salmonella was dependent on the concentration of gold nanoshells present in the system, which established a saturation limit for the gold nanoshells and helped form a working range for this Salmonella biosensor.

FIG. 10A shows the signal received by just HCl with no gold nanoshell input. A clear HCl peak at 0.6 volts can be observed from this image. FIG. 10B show the signal received from a mixture of gold nanoshells and HCl. A gold nanoshell peak and a diminished HCl peak can be observed at 0.3 volts and 0.6 volts, respectively. FIG. 10C shows the signal received from an oversaturated mixture of gold nanoshells and HCl. No clear peak can be seen for either gold or HCl in this case.

A sample containing 10 μL of stock gold nanoparticles (−2.2×10¹⁰ particles/mL) was diluted in 101 μL of 0.1 M HCl. This sample was hereafter be referred to as Dilution 1. A series of 1 in 10 Serial dilutions were then carried out. Dilution 2 was created using 1-part Dilution 1 and 9-parts 0.1 M HCl. Dilution 3 was created in a similar fashion. A blank sample, containing only 0.1 M HCl was always created. The final concentrations for each sample were 2.2×10⁹, 2.2×10⁸, 2.2×10⁷ particles per mL for Dilutions 1, 2, 3 and the blank sample respectively. Following dilution, 100 μL of each sample underwent square wave voltammetry. To do this, all samples were pre-conditioned at 1.3 volts for 30 seconds to oxidize all Au⁰ into Au³⁺. Following this, reduction of Au³⁺ was conducted by sweeping the potential from 0.15 volts to 0.6 volts, with a step potential of 0.004 volts, amplitude of 0.02 volts, and frequency of 100 Hz. The electrochemical signal from all samples were then recorded and this example was repeated four times.

The electrochemical signal from each dilution was recorded and a baseline signal was subtracted. Scans of the sample exhibited two peaks at roughly 0.3 volts and 0.6 volts with the former correlating with the presence of gold nanoshells. It can be seen by the similarity in signal received that Dilution 1 and Dilution 2 are samples that have oversaturated the biosensor. The sharper increase in signal for Dilution 3 is indicative that the sample no longer becomes saturated once the concentration of gold nanoshells drops below 2.2×10⁸ particles/mL. A summary of these results can be shown in Table 3.

TABLE 3

| | Electrochemical | Average Electrochemical | Coefficient |
|---|---|---|---|
| Sample ID | Signal Received (nA) | Signal (nA) | of Variance |
| Blank A | 54.376 | 66.422 | 19% |
| Blank B | 61.062 | | |
| Blank C | 66.249 | | |
| Blank D | 84 | | |
| Dilution 1 A | 107.812 | 112.234 | 8% |
| Dilution 1 B | 124.813 | | |
| Dilution 1 C | 110.438 | | |
| Dilution 1 D | 105.875 | | |
| Dilution 2 A | N/A | 111.229 | 11% |
| Dilution 2 B | 113.626 | | |
| Dilution 2 C | 98.5 | | |
| Dilution 2 D | 121.562 | | |
| Dilution 3 A | 142.25 | 133.531 | 14% |
| Dilution 3 B | 148.437 | | |
| Dilution 3 C | 136.125 | | |
| Dilution 3 D | 107.312 | | |

### EXPERIMENT 7

### Salmonella Dynabead™ Attachment Test

The efficiency of magnetic bead attachment for two different Salmonella typhimurium strains, ATCC 53647 and ATCC 53648 was investigated to determine if a different strain would result in improved attachment efficiency. Attachment of Salmonella to the anti-Salmonella magnetic Dynabeads™ was tested by plating the bacteria samples before and after attachment. To avoid nonspecific binding, the nonspecific binding sites were blocked in the centrifuge tubes used to process the samples with Pluronic® blocking agent. Pluronic® blocking agent is also available through Fisher Scientific. The samples were incubated in Difco™ nutrient broth for 24 hours at 37° C. A dilution series was used because using less dilute pure Salmonella samples resulted in plates with too many CFUs to count. Salmonella samples with an OD600 reading of 0.1 were diluted five times in a 10-fold dilution series. Samples were plated by spreading 50 μL of sample on Difco™ agar plates and incubating at 37° C. for 24 hours.

There were significant discrepancies in plate counts when plating the samples. For example, three plate counts taken from one bacteria sample had a CV of 16% (counts: 3.8×10⁷, 4.2×10⁷, 2.8×10⁷). Based on this result and other observed discrepancies, it was believed that at an incubation time of 24 hours, a portion of the bacteria may have been in death phase. The incubation time was shortened to 6 hours when the cells are believed to be a growth phase and repeated bead attachment efficiency experiments. While not wanting to be bound by any particle theory, it is believed that despite the manufacturer recommendations that the Dynabeads™ do not attach to Salmonella strain ATCC 53648 using PBS Buffer. These experiments have been repeated using 1× Borate Buffer Saline (BBS) Pierce™ 28341 (Rockford, Ill.) and have achieved higher attachment efficiencies. See Table 4. Using centrifuge tubes with the non-specific binding sites blocked and incubating the frozen Salmonella stock for 6 hours the attachment efficiency was 0% for all samples.

TABLE 4

| | Bead Attachment | Attachment | |
|---|---|---|---|
| 10-Fold Dilution Series | (CFU/mL) | Efficiency | CV |
| Dilution Series 4 | 0.00 | 0% | N/A |
| Dilution Series 4 | 0.00 | 0% | |
| Dilution Series 4 | 0.00 | 0% | |
| Dilution Series 5 | 0.00 | 0% | N/A |
| Dilution Series 5 | 0.00 | 0% | |
| Dilution Series 5 | 0.00 | 0% | |

*Note:
Bacteria sample measured OD600 = 0.08, Centrifuge tubes blocked with Pluornic and incubated for 8 hours.

Plate counts for Salmonella strain 53647 is believed to indicate that attachment is 5-7% when incubating the Salmonella stock for 24 hours. See Table 5.

TABLE 5

| | Bead Attachment | Attachment | |
|---|---|---|---|
| 10-Fold Dilution Series | (CFU/mL) | Efficiency | CV |
| Dilution Series 4 | 1.40 × 10⁶ | 4.6% | N/A |
| Dilution Series 5 | 2.00 × 10⁶ | 6.5% | N/A |

*Note:
Sample measured OD600 = 0.1, Centrifuge tubes blocked with Pluornic and incubated for 24 hours.

The experiment was repeated with an incubation time of 6 hours for improved attachment efficiency when the attachment is not affected by bacteria apoptosis. See Table 6. It was observed that under these conditions that a reduction in incubation time did not lead to improved attachment efficiencies.

TABLE 6

| | Bead Attachment | Attachment | |
|---|---|---|---|
| 10-Fold Dilution Series | (CFU/mL) | Efficiency | CV |
| Dilution Series 4 | 8.00 × 10⁵ | 5% | 53% |
| Dilution Series 4 | 4.00 × 10⁵ | 3% | |
| Dilution Series 4 | 2.00 × 10⁵ | 1% | |
| Dilution Series 5 | 0.00 | 0% | N/A |
| Dilution Series 5 | 2.00 × 10⁵ | 3% | |
| Dilution Series 5 | 0.00 | 0% | |

*Note:
Bacteria sample measured OD600 = 0.1, Centrifuge tubes blocked with Pluornic and incubated for 8 hours.

### EXAMPLE 8

### Magnetic Concentration for Amplification of Electrochemical Signal

When molecules are in low concentrations in a fluid, the concentration gradient present in the fluid may cause diffusion of the molecules across the entire fluid. Since gold nanoshells used in the detection protocol may be in low concentrations, the nanoshells may have a tendency to diffuse over the entire liquid. Such diffusion may be counterproductive when trying to detect a low level of pathogen since electron transfer may only occur in the diffusion layer of a fluid, where molecules are close to the electrode surface. To combat this, magnets to concentrate the Magnetic Bead-Pathogen-Gold nanoshells complexes near the surface of the electrode may be used. See the comparison between FIGS. 5A and 5B.

In an experiment, two 4 mL samples, sample A and B, containing roughly 5000 CFU/mL of E. coli were created. Four 1 mL aliquots of each sample were then collected. 10 μL of anti-E. coli complexed magnetic beads were then mixed in each aliquot for 40 minutes. Non-target particles were then washed out of the samples by performing immunomagnetic separation twice on each aliquot. 3 μL of antibody complexed gold nanoshells were then allowed to mix with each aliquot for 40 minutes. Immunomagnetic separation was then performed again twice to wash away any unbound gold nanoshells. Each aliquot was resuspended in 100 μL of 0.1 M HCl to concentrate the sample. Samples were then placed in their respective electrochemical well. Afterwards, magnetic concentration was performed on Sample A for 30 seconds per well while sample B was allowed to stay diffuse. Square wave voltammetry was performed on each well. A precondition voltage of 1.3 volts was applied for 30 seconds to pre-oxidized Au⁰ to Au³⁺. Following this, reduction of Au³⁺ was conducted by sweeping the potential from 0.15 volts to 0.6 volts, with a step potential of 0.004 volts, amplitude of 0.02 volts, and frequency of 100 Hz. The electrochemical signal from all samples were then recorded.

It was observed that the samples which had undergone magnetic concentration in the electrochemical well displayed not only an increase in electrochemical signal but also a reduction in well to well signal variation. This is likely because the magnetic concentration allows the majority of gold nanoshells present in solution to be reduced. Table 7 summarizes the results of this experiment.

TABLE 7

| | Electro- | Average | Co- |
|---|---|---|---|
| | chemical | Electro- | efficient |
| | Signal | chemical | of |
| Sample ID | Received (nA) | Signal (nA) | Variance |
| Sample A (magnetically | 107 | 107.975 | 2% |
| concentrated AuNS complexes 1) | | | |
| Sample A (magnetically | 106 | | |
| concentrated AuNS complexes 2) | | | |
| Sample A (magnetically | 107.7 | | |
| concentrated AuNS complexes 3) | | | |
| Sample A (magnetically | 111.2 | | |
| concentrated AuNS complexes 4) | | | |
| Sample B (diffuse AuNS | 72.8 | 96.675 | 23% |
| complexes 1) | | | |
| Sample B (diffuse AuNS | 85.3 | | |
| complexes 2) | | | |
| Sample B (diffuse AuNS | 106.9 | | |
| complexes 3) | | | |
| Sample B (diffuse AuNS | 121.7 | | |
| complexes 4) | | | |

### EXAMPLE 9

### Detection of E. coli

In this example, four 3 mL samples, A, B, C, and D, were created. Sample A contained 0 CFU/mL of E. coli as a negative control. Sample B contained 5000 CFU/mL of E. coli. Sample C contained 500 CFU/mL of E. coli. Sample D contained 50 CFU/mL of E. coli. Three 1 mL aliquots of each sample were then collected, and a bio-sensing protocol was conducted as described previously. The electrochemical signal from all samples were then recorded.

There was an observable difference for all levels (5000, 500, and 50 CFU/mL) from the blank sample using the biosensing protocol. However, considerable variability was observed in some of the samples. Without being bound to any particular theory, this variation is believed to be due to the magnetic concentration step. The magnet used to concentrate samples in this example was not applied in a uniform fashion to all samples, which is believed to have resulted in some samples being properly concentrated on the surface of the electrode as desired and some samples not being concentrated in the desired location. Table 8 summarizes the results received from this experiment.

TABLE 8

| | Electro- | Average | |
|---|---|---|---|
| | chemical | Electro- | |
| | Signal | chemical | Coefficient |
| Sample ID | Received (nA) | Signal (nA) | of Variance |
| Sample A (0 CFU/mL 1) | 104.251 | 114.773 | 8% |
| Sample A (0 CFU/mL 2) | 123.503 | | |
| Sample A (0 CFU/mL 3) | 116.565 | | |
| Sample B (5000 CFU/mL 1) | 135.377 | 134.148 | 15% |
| Sample B (5000 CFU/mL 2) | 113.19 | | |
| Sample B (5000 CFU/mL 3) | 153.877 | | |
| Sample C (500 CFU/mL 1) | 124.064 | 143.0965 | 19% |
| Sample C (500 CFU/mL 2) | 162.128 | | |
| Sample C (500 CFU/mL 3) | N/A | | |
| Sample D (50 CFU/mL 1) | 111.626 | 121.876 | 8% |
| Sample D (50 CFU/mL 2) | 129.689 | | |
| Sample D (50 CFU/mL 3) | 124.314 | | |

### EXAMPLE 10

### Signal Testing in Pathogen Detection

One challenge when building an automated pathogen detection system that is to be deployed in areas remote from modern civilization is providing a result that can be easily interpreted by personnel with minimal training. This is difficult due to the complexity of each assay run for each pathogen detected, natural variability present in each sample, and any human error in sample prep/sample loading steps. The ability to differentiate from a true negative and a false negative is especially pronounced when dealing with dilute samples that have a rare incidence of a desired pathogen. In this example, use of a normalized difference between electrochemical scans was investigated.

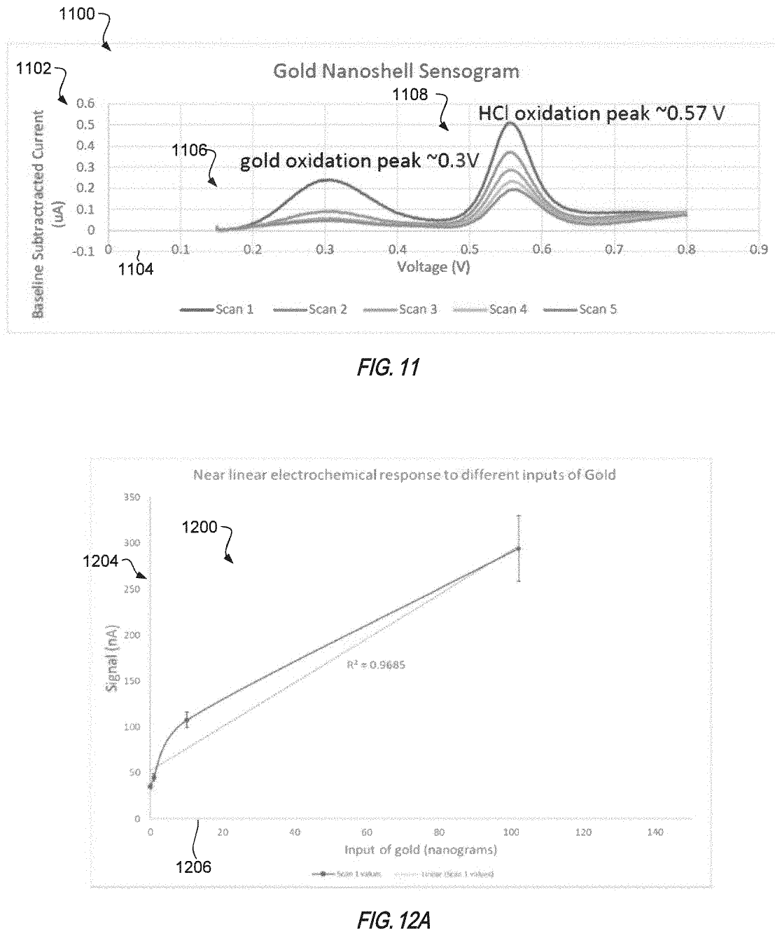

FIG. 11 depicts a chart 1100 with the results of the square wave voltammetry of a sample. The y-axis 1102 represents a measurement of electrical current, and the x-axis 1104 represents a voltage applied to the electrode. In this example, two Redox peaks are observed. The first peak 1106 indicates the oxidation of gold from Au⁰ to HAuCl⁴ which occurs at approximately 0.3 volts. The second peak 1108 is indicative of the redox of HCl which occurs at approximately 0.57 volts. A typical nonpathogenic sample was electrochemically scanned four times and the signal strength for all peaks was recorded for each scan. When the second through fourth electrochemical scans were performed, a noticeable drop in signal strength for both the HCl redox peaks and the gold nanoshell peaks were observed. It has also been observed that the difference in these peaks is dependent on the concentration of gold nanoshell present in a sample, being more drastic when gold nanoshell are completely absent in a sample (such as a blank sample).

In some cases, when a consistent relationship for gold signal strength between samples of varying concentration can be determined, then a normalized difference signal between 0 and 1 can be used to denote true presence/absence of gold in this system. This normalized signal may involve the subtraction of the gold redox peak recorded in the fourth scan from the gold peak recorded in the first scan divided by the value of the peak in the first scan. This may be represented with the following normalized signal equation.

(Scan 1−Scan 41)/Scan 1

### EXAMPLE 11

### Optimization of Electrochemical Detection

The ability to differentiate between a true negative and a false negative sample becomes more challenging when the pathogen materials in a sample are at very low concentration. Another method to combat the occurrence of a false negative is to widen the gap between electrochemical signal received by very low concentration of gold and electrochemical signals received in the absence of gold. In this example, the input parameters to conduct square wave electrochemistry were varied to maximize the difference between the electrochemical signal received from Scan 1 (presence of gold) and Scan 4 (absence of gold).

In this example, four 412 μL samples, A, B, C, and D, were created. Each sample contained 12 μL of gold nanoshells diluted in 400 μL of 0.1 M HCl. Four 103-μL aliquots of each sample were then collected. Each aliquot underwent square wave voltammetry with different input parameters. Aliquots from sample A underwent square wave voltammetry using the historical parameter which all previous tests have used. Aliquots from sample B underwent square wave voltammetry using a step potential (E-step) of 0.002 volts and a frequency of 100 Hz. Aliquots from sample C underwent square wave voltammetry using a step potential (E-step) of 0.0004 volts and a frequency of 100 Hz. Aliquots from sample D underwent square wave voltammetry using a step potential (E-step) of 0.0004 volts and a frequency of 50 Hz. All other parameters were kept constant for all aliquots. The normalized signal from each sample was then recorded.

During this experiment, it was observed that some settings used to conduct square wave voltammetry produced the smallest normalized signal as well as the greatest variability between samples. While not wanting to be bound to any particular theory, this may be due to an incomplete reduction of gold using these settings. Parameter set B and parameter set C both resulted in high normalized signal and limited variance between aliquots. Table 9 and Table 10 contain a summary of the results of this study.

TABLE 9

| | | | Average | Co- |
|---|---|---|---|---|
| | | Signal | signal | efficient |
| Parameter | Parameters | received | received | of |
| set name | varied | (nA) | (nA) | Variance |
| Historical settings, | E-step: 0.004 V | 258.62 | 244.67 | 8% |
| Parameter set A | Frequency: | 221.13 | | |
| | 100 Hz | 254.25 | | |
| Historical settings, | E-step: 0.002 V | 242.24 | 274.73 | 22% |
| Parameter set B | Frequency: | 236.77 | | |
| | 100 Hz | 345.19 | | |
| Historical settings, | E-step: 0.0004 V | 275.66 | 264.75 | 8% |
| Parameter set C | Frequency: | 277.37 | | |
| | 100 Hz | 241.23 | | |
| Historical settings, | E-step: 0.0004 V | 188.31 | 214.18 | 11% |
| Parameter set D | Frequency: | 237.24 | | |
| | 50 Hz | 216.83 | | |

TABLE 10

| | | Normalized | Average | | Time |
|---|---|---|---|---|---|
| Parameter set | Parameters | Signal | signal | Coefficient | required per |
| name | varied | received (nA) | received (nA) | of Variance | test (minutes) |
| Historical | E-step: 0.004 V | 0.6191 | 0.5623 | 13% | 1.25 |
| settings, | Frequency: | 0.4949 | | | |
| Parameter set A | 100 Hz | 0.5049 | | | |
| Historical | E-step: 0.002 V | 0.6974 | 0.6675 | 7% | 1.4 |
| settings, | Frequency: | 0.6304 | | | |
| Parameter set B | 100 Hz | 0.6299 | | | |
| Historical | E-step: 0.0004 V | 0.7936 | 0.7969 | 1.2% | 2.5 |
| settings, | Frequency: | 0.8074 | | | |
| Parameter set C | 100 Hz | 0.7895 | | | |
| Historical | E-step: 0.0004 V | 0.9063 | 0.90 | 0.2% | 4 |
| settings, | Frequency: | 0.9079 | | | |
| Parameter set D | 50 Hz | 0.9094 | | | |

### EXAMPLE 12

### Listeria and Salmonella Dynabead™ Attachment Tests

In this example, the efficiency of magnetic bead attachment for Salmonella typhimurium, strain ATCC 53647 and Listeria innocua, ATCC 33090 was tested. Attachment of bacteria to the anti-Salmonella and anti-Listeria magnetic Dynabeads™ was tested by plating the bacteria samples before and after attachment. Initially samples were incubated for 24 hours at 37° C. in Difco™ Nutrient Broth and Difco™ Brain and Heart Infusion broth, respectively. A dilution series was used because using less dilute pure bacteria samples resulted in plates with too many CFUs to count. Bacteria samples with an OD600 reading of 0.1 were diluted six times in a ten-fold dilution series. Samples were plated by spreading 50 μL of sample on Difco™ agar plates and incubating at 37° C. for 24 hours. It was observed that the attachment of Salmonella and Listeria to magnetic beads is sensitive to the temperature of the reagents used. Reagents and agar media plates stored at 2° C. were brought to room temperature before use.

Table 11 and Table 12 depict results for this example. The plate counts for Salmonella strain 53647 that indicate that attachment is 22-49% when increasing reagent temperatures to room temperature before use.

TABLE 11

| | Bead Attachment | Attachment | |
|---|---|---|---|
| 10-Fold Dilution Series | (CFU/mL) | Efficiency | CV |
| Dilution Series 6 | 14 | 46% | N/A |
| Dilution Series 6 | 12 | 48% | N/A |
| Dilution Series 6 | 4 | 22% | N/A |

*Note:
Sample measured OD600 = 0.1

Plate counts for Listeria innocua, ATCC 33090 were 12-17%.

TABLE 12

| | Bead Attachment | Attachment | |
|---|---|---|---|
| 10-Fold Dilution Series | (CFU/mL) | Efficiency | CV |
| Dilution Series 6 | 8 | 14% | N/A |
| Dilution Series 6 | 7 | 12% | N/A |
| Dilution Series 6 | 10 | 17% | N/A |

*Note:
Sample measured OD600 = 0.1

### EXAMPLE 13

### Gold Nanoparticle Detection

In some examples, the electrochemical sensor may reach a saturation point in which the response received from the sensor no longer increases linearly with the nonmagnetic metal input to the sensor. A linear response may affirmatively differentiate different levels of pathogens detected in the system. In this example, nine samples containing a total volume of 103 μL of a mix of 0.1 M HCl and various concentrations 150 nm gold nanoshells were created. These samples contained 2040,1020, 510, 255, 127.5, 102, 10.2, 1.02, and 0 nanograms of gold respectively. Quadruplets of each sample were placed in their respective electrochemical well, and 4 square wave voltammetric scans were run for each sample using the following settings:

| | |
|---|---|
| Pretreatment voltage (Scan 1) | 1.3 V |
| Pretreatment time (Scan 1) | 30 seconds |
| Equilibrium time between scans | 8 seconds |
| SWV beginning voltage | 0.15 V |
| SWV ending voltage | 0.8 V |
| Voltage size step | 0.0004 V |
| Amplitude | 0.02 V |
| Frequency | 100 Hz |

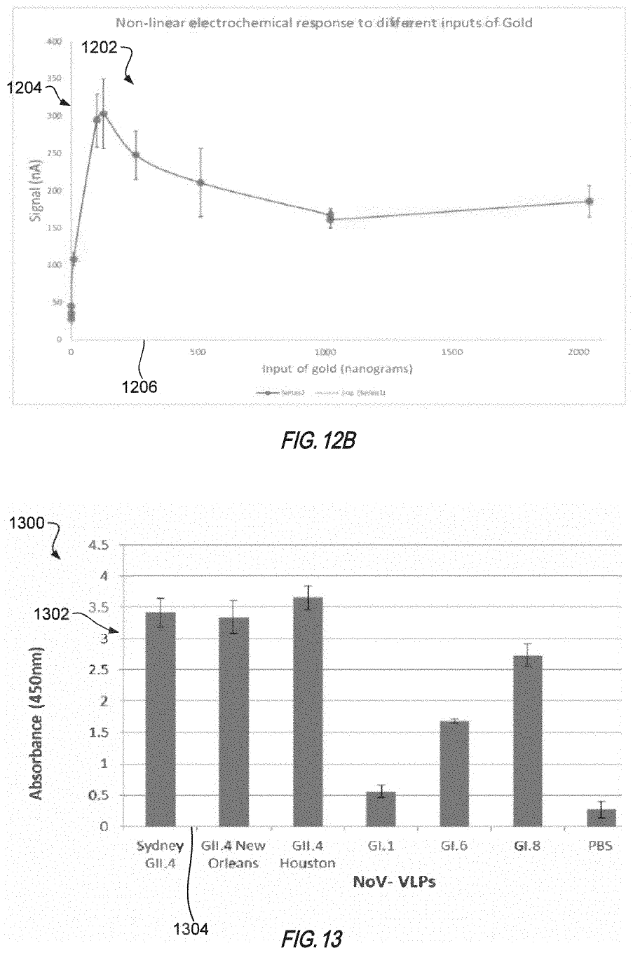

It was observed that the electrochemical detection system exhibited an almost linear response for inputs below 102 nanograms of gold. Saturation quickly occurs after input of 102 nanograms as a nonlinear response was observed when 127.5 nanograms of gold was input. Under these parameters, the amount of gold that can be complexed with bacteria and still get a linear response from the electrochemical sensor may approach the 102 nanograms of gold. However, under different circumstances, the saturation point may be achieved with a different amount of nonmagnetic metal. FIGS. 12A and 12B depicts charts 1200 and 1202 that illustrate the linear and nonlinear behavior of this electrochemical sensor at different inputs of gold of this experiment. Chart 1200 depicts the linear response, and chart 1202 depicts the nonlinear response. In these figures, the y-axis 1204 represents the signal measured in current, and the x-axis 1206 represents an input of gold on the nanoscale.

### EXAMPLE 14

### Antibody Binding and Magnetic Bead Immunocapture

This example sought to develop and validate a protocol for complexing antivirus antibody to magnetic beads and to evaluate magnetic bead immunocapture efficiencies for Hepatitis A and Norovirus. In this example, the reactivity of monoclonal antibody (Maine Biotechnology #MAB 242P) was evaluated on norovirus virus like particles (VLP) showing broad reactivity across groups I and II of human norovirus indicating that the antibody is effective in capture of group 1 and 2 norovirus. A biotin conjugation kit (Abcam #ab201795) was used to complex anti-norovirus antibody (Maine Biotechnology #MAB 242P). The antibody was then coupled to streptavidin coated magnetic beads (Dynabeads M-280 Invitrogen #11205D). The 50 μL of the prepared beads (10 mg/mL) were then incubated with a serial dilution of norovirus for 2 hours to capture free norovirus. Following the wash tasks and immunomagnetic separation, ribonucleic acid (RNA) was extracted and analyzed with quantitative reverse transcription polymerase chain reaction (qRT-PCR). This process was repeated for Hepatitis A virus. FIG. 13 depicts a chart 1300 of the results of these experiment. In this example, y-axis 1302 represents a wavelength absorption, and the x-axis 1304 represents Norovirus virus-like particles.

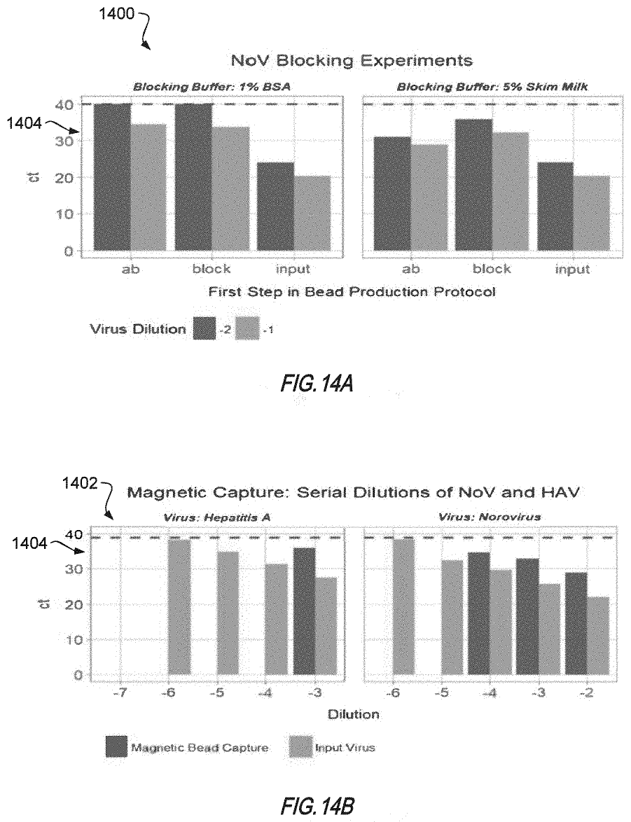

The plots 1400, 1402 in the FIGS. 14A and 14B use a threshold cycle (ct) value represented in the y-axis 1404. A low ct value indicates that a high number of copies of virus nucleic acid were present. A high ct value (near 40) indicate samples with very low nucleic acid. As such, lower ct values in the plots below indicate that more virus was recovered and thus had a higher efficiency. Following qPCR amplification of the bound nucleic acids, approximate bead capture efficiencies were determined. A dilution of −4 is roughly equivalent to 1000 viruses/mL, which calculates to a capture efficiency of 1-2% at a −4 dilution.

### EXAMPLE 15

### Bacteria Detection

Table 13 represents an additional experiment for bacterial detection with Salmonella in biosafety level 2 (BSL 2) facilities. In this experiment, the antibodies were designed specifically for these BSL 2 strains, and immunocapture efficiencies close to 50% with about 8% variation were obtained.

TABLE 13

| | | Dilution | CFU/ | | |
|---|---|---|---|---|---|
| Plate | Count | Factor | mL | Mean | SD |
| 7A | 13 | 1 | 13 | 14.66667 | 1.247219 |
| 7B | 15 | 1 | 15 | | |
| 7C | 16 | 1 | 16 | | |
| Negative Control | 0 | 1 | 0 | | |
| D7 Control 1 | 28 | 1 | 28 | 29.66667 | 9.46338 |
| D7 Control 2 | 19 | 1 | 19 | | |
| D7 Control 3 | 42 | 1 | 42 | | |
| 7A Wash | 1 | 20 | 20 | 13.33333 | 9.42809 |
| 7B Wash | 1 | 20 | 20 | | |
| 7C Wash | 0 | 20 | 0 | | |

| Individual Recovery | Average Recovery |
|---|---|
| 0.438202247 | 0.494382022 |
| 0.505617978 | |
| 0.539325843 | |

### EXAMPLE 16

### Automation

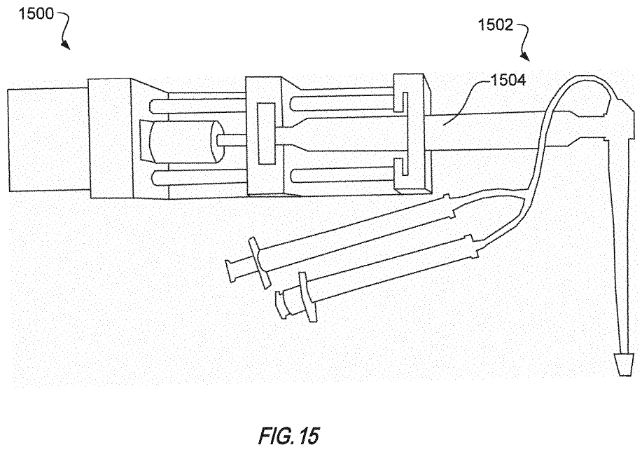

FIG. 15 depicts an example of a syringe pump 1500 built for this experiment delivers microliter doses of magnetic beads and gold nanoparticles. The syringe pump 1500 includes a 3D printed pipette tip adaptor 1502 that can add solutions to a centrifuge tube 1504 and that can remove waste and pipette mixes. At least some of the components that contact the fluid are either disposable (syringes, pipette tips, capillary tubes) or can be cleaned by flushing water and ethanol through the system. Fluid can be moved through the system using syringe pumps. Multiple pumps may be combined with their respective syringes to move fluids with the magnetic beads, gold nanoparticles, buffer solutions, samples, and so forth. The pumps may cause the fluids to be mixed by providing negative and positive pressure to the pipette tip adaptor. The pump compresses the syringe plunger providing positive pressure and retracts the syringe plunger creating suction. While FIG. 15 depicts just a single syringe connected to a pump, each of the syringes may be connected to their respective pumps to control the fluids within the volumes of their syringes.

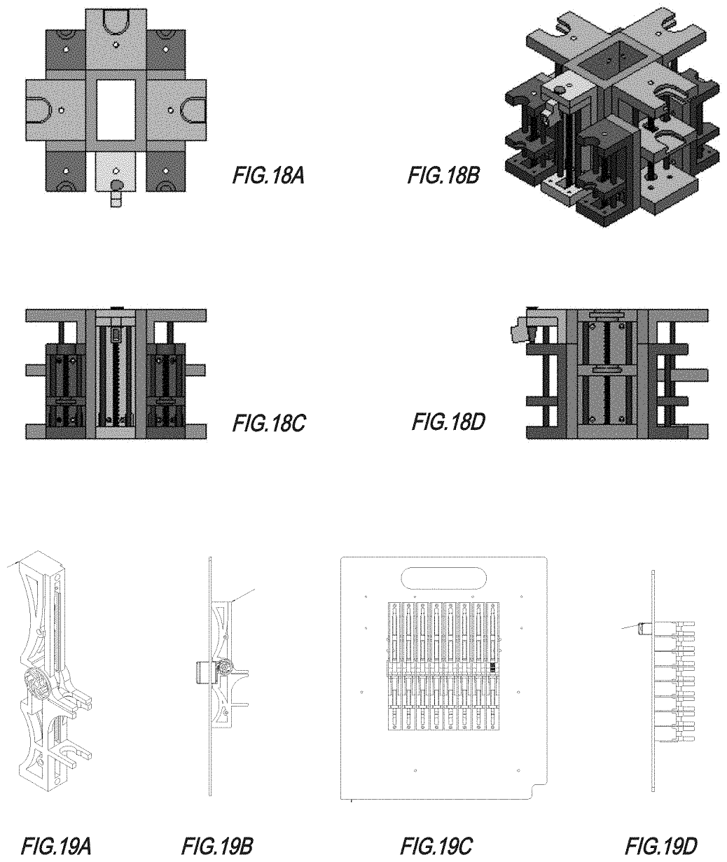

Automated sample processing devices were used to perform a benchtop protocol for the formation of antibody-magnetic-bead/pathogen/antibody-gold-nanoparticle (AB-MB/Pathogen/AB-AuNP) assays. Two versions of the device have been developed and are illustrated in FIGS. 18A-D and 19A-D. The first version of the device, illustrated in FIGS. 18A-D utilized stepper motor driven syringe pumps that allowed for variable measurements of reagents. The second device, illustrated in FIGS. 19A-D was comprised of servo driven linear actuated syringe pumps that deliver fixed volumes of reagents. In an exemplary, non-limiting example of their use, bacterial samples of various concentrations, suspended in 500 μL of 1×PBS, 0.05% w/v BSA and 0.05% v/v Tween® 20, could be loaded into a 1.7 mL centrifuge tube and incubation steps performed by aspirating and expelling the suspended sample through a filtered pipette tip (i.e. pipette mixing). Waste removal, reagent delivery, and magnetic separation could be performed using syringe pumps and linear actuators driven by stepper motors (FIGS. 18A-D) and continuous servo motors (FIGS. 19A-D).

A tube may direct fluid out of the pipette tip adaptor and drop liquid into the tip. Pressure generated by syringe pumps may drive the fluid through the pipette tip into a centrifuge tube using air after the fluid has left the syringe. A second syringe pump may be connected to the tube, also connected to another syringe pump to move buffer solution into the centrifuge tube during immuno-magnetic separation wash cycles.

At least one of the pumps may cause a withdrawal of effluent from the centrifuge tube through a disposable glass capillary tube that extends near the inlet of the pipette tip. The capillary tube may have a small surface area limiting the occurrence of nonspecific binding to the outer surface.

In some cases, a procedure for mixing and magnetic separation may include adding samples and magnetic beads through a first syringe pump, mixing in the pipette with a second syringe pump, and placing a magnet against the centrifuge tube wall to fix the magnetic beads inside tube. Next, the magnetic separation effluent may be removed with the second syringe pump through the capillary tube, adding buffer solution through the second syringe pump, and removing the magnet and resuspend solution by pipette mixing. At least some of the tasks in this procedure may be repeated multiple times. Then, adding the gold nanoshells with the buffer solution. and repeating portions of the method involving suspending the magnetic beads with the magnets while cleaning the sample. Next, the solution may be transferred to at least one electrochemical well plate.

In some cases, the syringe pumps may be calibrated by transferring fluid onto a scale and measuring the weight of the transferred fluid. Samples may then be collected on a cotton swab inside a centrifuge tube for ease of measurements and to avoid evaporation skewing experimental results. The syringe pumps may operate with a stepper motor driving a precision lead screw, turning 1.8-inch step or 200 steps per revolution. The lead screw may have a linear motion of 1/20.8″ per revolution and a 1 mL syringe may have an inner diameter (D) of 4.71 millimeter. In this case, the resolution of the syringe pump may be calculated as (linear motion/revolution)/(steps/revolution)*(πD2)/4=(volume/step) or as 0.106 microliter/step. In one experiment, the measured resolution of the syringe pump was found to be 0.050-0.052 microliter/step, which is recorded in Table 14. The discrepancy between measured and calculated values indicates, syringe pumps administering the magnetic beads or gold nanoparticles may need to be calibrated to ensure accurate measurements. The repeatability of measurements and fine resolution indicate the syringe pumps may effectively dispense microliter size doses.

TABLE 14

| # | Initial | Final | Pumped | Average Pumped |
|---|---|---|---|---|
| Steps | Weight (g) | Weight (g) | Weight (g) | Weight (g) |
| 10 | 1.03168 | 1.03228 | 6.00E−04 | 5.14E−04 |
| 10 | 1.03227 | 1.03284 | 5.70E−04 | |
| 10 | 1.03287 | 1.03333 | 4.60E−04 | |
| 10 | 1.03337 | 1.03378 | 4.10E−04 | |
| 10 | 1.03376 | 1.03429 | 5.30E−04 | |
| 100 | 1.03429 | 1.0389 | 4.61E−03 | 5.06E−03 |
| 100 | 1.03896 | 1.0436 | 4.64E−03 | |
| 100 | 1.0436 | 1.04904 | 5.44E−03 | |
| 100 | 1.04904 | 1.05497 | 5.93E−03 | |
| 100 | 1.05497 | 1.05964 | 4.67E−03 | |
| 1000 | 1.05963 | 1.11197 | 5.23E−02 | 5.19E−02 |
| 1000 | 1.11198 | 1.16415 | 5.22E−02 | |
| 1000 | 1.16415 | 1.21575 | 5.16E−02 | |
| 1000 | 1.2157 | 1.26784 | 5.21E−02 | |
| 1000 | 1.26786 | 1.31935 | 5.15E−02 | |

### EXAMPLE 17

### Electrochemical Detection of Gold Nanoshells Bound to Magnetic Beads

In some cases, running electrochemistry of gold in solution is not as accurate of a measure for the sensor response because the sensor may not react with the whole solution. Directly binding gold nanoshells to magnetic beads may provide the ability to magnetically concentrate a known mass of gold nanoshells to the electrode. This may allow us to set a baseline for gold detection which can be compared to samples containing pathogens.

One such method may include the following tasks:
1. Prepare EDC and Sulfo-NHS at 10 mg/mL in H₂O immediately before forming the complexes.
2. Add 8 μL EDC to 1 mL of BioReady™ 150 nm Carboxyl Gold Nanoshells.
3. Add 16 μL Sulfo-NHS to the 1 mL of BioReady™ 150 nm Carboxyl Gold Nanoshells.
4. Vortex solution then incubate at room temperature for 30 minutes while rotating.
5. Centrifuge at 2000 RCF for 5 minutes.
6. Carefully remove supernatant to remove any excess EDC/Sulfo-NHS and resuspend pelleted nanoparticles with 1 mL of Reaction Buffer. Sonicate or vortex<30 seconds.
7. Add 1 mg of biocytin and vortex.
8. Incubate at room temperature for 40 minutes while rotating.
9. After incubation, add 5 μL of quencher to deactivate any remaining active NHS-esters. Vortex an incubate at room temperature for 5 minutes while rotating.
10. Centrifuge at 2000 RCF for 5 minutes. Carefully remove supernatant and resuspend pellet with 1 mL Reaction Buffer. Sonicate<30 seconds.
11. Repeat task 10.
12. Centrifuge again at 2000 RCF for 5 minutes. Remove supernatant and resuspend pellet in 1 mL of 1×PBS. Sonicate<30 seconds.
13. Resuspend the Dynabeads™ M-280 Streptavidin in the vial (i.e. vortex for >30 sec, or tilt and rotate for 5 min).
14. Transfer 40 μL of Dynabeads™ M-280 Streptavidin to a 1.5 mL tube.
15. Add 1 mL of phosphate buffered saline and mix (vortex for 5 seconds or keep on a roller for at least 5 min).
16. Place the tube on a magnet for 1 minute and discard the supernatant.
17. Remove the tube from the magnet and resuspend the washed Dynabeads™ in 100 μL of PBS.
18. Add 100 μL of washed Dynabeads™ M-280 Streptavidin to the prepared BioReady™ 150 nm Carboxyl Gold Nanoshells.
19. Incubate the Streptavidin coated Dynabeads™ and the biotinylated Gold Nanoshells for 30 minutes at room temperature using gentle rotation.
20. Separate the antibody-coated beads with a magnet for 3 minutes.
21. Wash the coated beads 4-5 times in 0.1× phosphate buffered saline containing 0.1% BSA.
22. Resuspend to the desired concentration for your application.

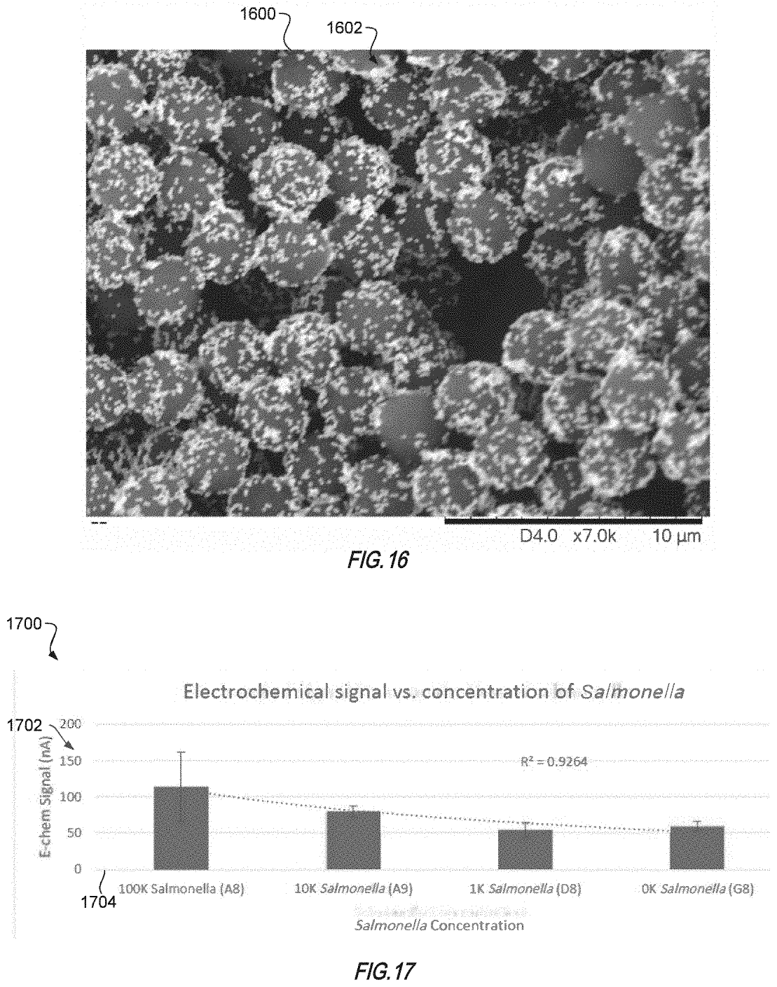

Additionally, inductively coupled plasma mass spectrometry may be used to quantify the amount of gold bound to magnetic beads. In some experiments, this value was shown to be roughly 0.2 nanograms of gold per nanoliter of stock magnetic beads. FIG. 16 depicts an example of nanoshells bound to magnetic beads. FIG. 16 depicts a SEM image of gold nanoshells 1600, which are the smaller white spheres bound to magnetic beads 1602, which are the larger spheres.

### EXAMPLE 18

### Electrochemical Detection of Salmonella Using Gold Nanoshells

In this example, anti-Salmonella antibody complexed with 150 nm Gold nanoshells were created using via a carboxyl to amine group conjugation chemistry. A dilution series of heat killed BacTrace® Salmonella was created. From this dilution series, four samples, A, B, C, and D, were created. These samples contained approximately 100,000, 10,000, 1000, and 0 colony forming units of Salmonella/mL diluted in PBS, respectively. Five hundred μL of each sample were mixed with 10 μL of anti-Salmonella coated magnetic beads. Samples were allowed to rotate end over end for 20 minutes each Immunomagnetic separation was then performed again twice to wash away any unwanted debris in the system. 3 μL of antibody complexed with gold nanoshells were then allowed to mix with each aliquot for 35 minutes. Immunomagnetic separation was then performed again twice to wash away any unbound gold nanoshells. Each aliquot was resuspended in 100 μL of 0.1 M HCl to concentrate the sample. Samples were then placed in their respective electrochemical well. Afterwards, magnetic concentration was performed on all samples and square wave voltammetry was performed on each well. Quintuplets of each sample were tested, and signals identified as outliers were ignored.

A distinguishable electrochemical signal was observed for the samples containing 100,000 and 10,000 CFU/mL of Salmonella. The 1000 CFU/mL sample was indistinguishable from the blank sample. In previous experiments, the signals received for the blank samples were 30-40 nanoamps. A summary of the results of this test can be found in FIG. 17. In FIG. 17, the chart 1700 includes a y-axis 1702 representing electrical current, and an x-axis 1704 representing the sample concentrations.

### EXAMPLE 19

### Automated Pipette Mixing Characterization

In this example, the ability of a new automated pipette mixing device was tested by performing an attachment efficiency test for E. coli O157:H7. This test was performed to investigate the ability of the automation device to mix the samples without the loss of attachment efficiency. The performance of the automated pipette mixer was confirmed by comparing capture efficiency of the device to that of mixing on a rotator.

In this example, the automated pipette was used to mix anti-E. coli (Dynabeads™ 71003) with a 3×10⁷-4×10⁷ CFU/mL sample. A simplified protocol for the capture of E. coli using 2.8-mircometer magnetic beads may include the following tasks:
1. Dilute a concentrated sample to an OD600 reading of 0.1 (correlating to approximately 3.0×10⁷ CFU/mL based on averaged plating results).
2. Dilute sample by in a 10-fold dilution series, 5 times.
3. Aliquot the final dilution into three 1 mL samples.
4. Add 20 μL of anti-Listeria beads to each sample.
5. Suspended on a rotating rack for 30 minutes.
6. Wash each sample using IMS and collect supernatant.
7. Repeat IMS wash for each sample and collect supernatant
8. Plate 100 μL of samples containing beads, a sample not containing beads and the collected supernatant washes.

Table 15 Capture efficiency test comparing automated pipette mixing to rotational mixing.

TABLE 15

| | Concentration (CFU/mL) | Capture Efficiency |
|---|---|---|
| Automated | 320 | 94% |
| Pipette Mixing | 100 | 100% |
| | 300 | 97% |
| Rotational | 320 | 97% |
| Mixing | 100 | 100% |
| | 400 | 95% |

### EXAMPLE 20

### Virus Detection

This example for forming complexes of aptamer to 15 nm magnetic nanoparticles included the following tasks:
1. Add 30 μL 15 nm magnetic nanoparticle stock to 1 mL phosphate buffered saline with Tween-20.
2. Recover beads by centrifugation and wash with 1 mL phosphate buffered saline with Tween-20 (1×).
3. Resuspend in 1 mL phosphate buffered saline with Tween-20.
4. Add 50 μL of biotinylated Aptamer/HBGA. Incubate on rotator for 1 hour at room temperature.
5. Recover beads by centrifugation and wash with 1 mL phosphate buffered saline with Tween-20 (1×).
6. Resuspend in blocking buffer: 1% skim milk in phosphate buffered saline with Tween-20.
7. Incubate beads on rotator overnight at 4 C.
8. Also block all tubes needed for experiment.
9. Recover beads by centrifugation and wash with 1 mL phosphate buffered saline with Tween-20 (1×).
10. Resuspend beads in 1 mL phosphate buffered saline with Tween-20.
11. Remove blocking buffer from extra tubes and wash with 1 mL phosphate buffered saline with Tween-20 (2×).

An additional experiment for Norovirus capture using aptamer complexed with 15 nm magnetic particle included the following tasks:
1) To blocked microcentrifuge tube add:
   a) 800 μL phosphate buffered saline with Tween-20
   b) 100 μL of virus sample
   c) 100 μL of complexed/blocked beads
2) Incubate 2 hours at room temperature in rotator.
3) Recover beads by centrifugation.
   a) Wash once with phosphate buffered saline with Tween-20
   b) Wash once with phosphate buffered saline
   c) Resuspend the beads in 500 μL of phosphate buffered saline (can vary to match any input controls).
4) Perform RNA extraction, followed by qRT-PCR for detection.

These tests indicated that there is a bead size may affect capture efficiency.

### EXAMPLE 21

### Capture Efficiency

During certain experiments, it was observed that specific and scrambled aptamer both appear to result in high capture efficiency. Additionally, it was also observed that the use of 1% skim milk as a blocking buffer increases capture efficiencies.

### EXAMPLE 22

### Salmonella Magnetic Bead Capture Efficiency

In this example, Dynabeads™ Anti-Salmonella (Catalog no. 71002) were used to capture Salmonella (ATCC 31194). Salmonella was prepared using standard technique and a dilution series was prepared in 1×PBS. IMS was completed using 20 μL of stock beads with an incubation time of 20 minutes followed by supernatant removal and 2 washes in 1×PBS. Bead samples were plated using standard techniques on agar medium #3. Plates were counted at 18-24 hours post plating. The IMS recovery of Salmonella (Catalog no. 71002) with Dynabeads™ Anti-Salmonella was observed to be near 50% at all dilutions.

### EXAMPLE 23

### Listeria spp. Capture Efficiency

During this example, Dynabeads™ were attached to Listeria spp. and the samples plated on agar plates to observe colony forming units (CFU). A simplified protocol for the capture of Listeria spp. using 2.8-micrometer magnetic beads included the following tasks:
1. Dilute a concentrated sample to an OD600 reading of 0.1 (correlating to 3.7×10⁶ CFU/mL based on averaged plating results).
2. Dilute sample in 10-fold dilution series, 5 times.
3. Aliquot each dilution into three 1 mL samples.
4. Add 20 μL of anti-Listeria beads to each sample.
5. Suspended on a rotating rack for 10 minutes.
6. Wash each sample using immuno-magnetic separation (IMS) and collect supernatant.
7. Resuspended each sample for an additional 10 minutes.
8. Repeat IMS wash for each sample and collect supernatant
9. Plate 100 μL of samples containing beads, a sample not containing beads and the collected supernatant washes.

Following this protocol, it was observed that the magnetic beads did not attach to Listeria samples with concentrations 1,880-2,200 CFU/mL. Capture efficiencies of approximately 10% were observed for concentrations between 2-4.3 CFU/mL. For concentrations between 4,470-16 CFU/mL the efficiency dropped to approximately 1.2-3.5%. The limit of attachment was observed to be near 380 CFU/mL based on experimental results, with 0% attachment at this concentration.

Table 16 includes results of the selectivity test for anti-Salmonella and streptavidin magnetic. It was observed that these beads held no affinity for attachment to Listeria under the parameters of this experiment.

TABLE 16

| | | Concentration | Capture |
|---|---|---|---|
| | Sample # | (CFU/mL) | Efficiency |
| Streptavidin Coated | 1 | 2060 | 0% |
| Magnetic Bead | 2 | 1740 | 0% |
| | 3 | 1860 | 0% |
| anti-Salmonella | 1 | 2220 | 0% |
| Magnetic Bead | 2 | 1920 | 0% |
| | 3 | 1880 | 0% |

### EXAMPLE 24

### Electrochemical Detection of Zikavirus

### Objective

Achieve electrochemical detection of Zikavirus capsid at limits of 1 to 10 ng/mL.

### Method

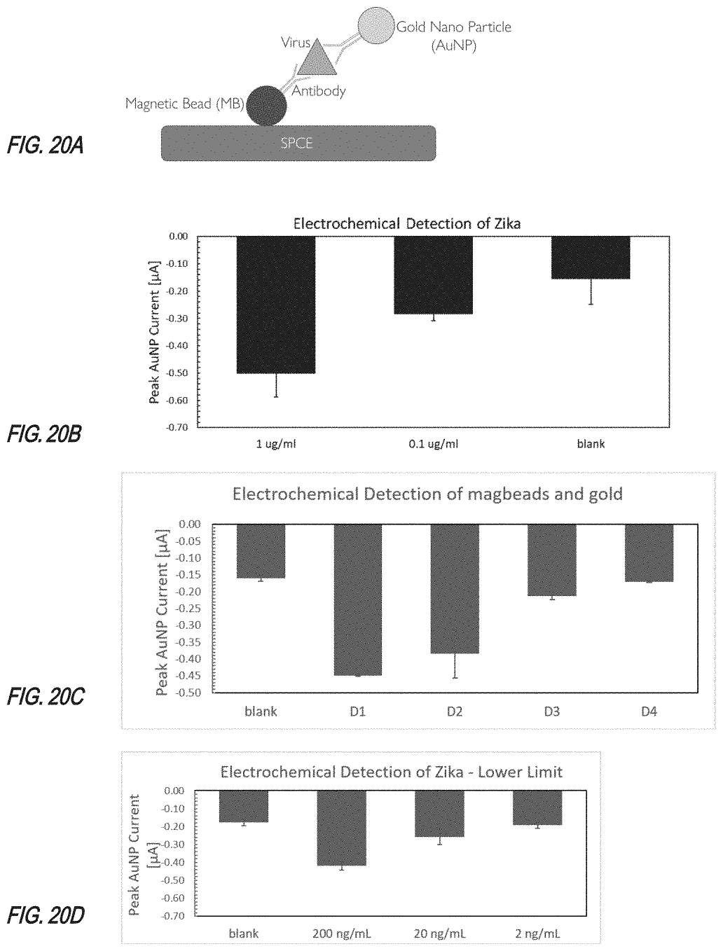

In summary, a sample containing Zika virus capsids was incubated with 150 nm magnetic nanoparticles (MNP) to capture the virus followed by incubation with 20 nm gold nanoparticles (AuNP) to create MNP-virus-AuNP complexes (shown in FIG. 20A). The complexes were then concentrated using immunomagnetic separation and placed on a screen-printed carbon electrode (SPCE). The complexes are magnetically captured to the surface of the SPCE using a magnet. The sample was analyzed electrochemically using a potentiostat where the signal correlated directly with the number of AuNP on the electrode surface.

### Results & Discussion

The detection results for 500 μL samples containing Zika capsid are shown in FIG. 20B. The 1 μg/mL and blank samples are a replicate of 3 while the 0.1 μg/mL samples have a replicate of 4. Results indicated that the limit of detection of 100 ng/mL. We explored potential causes for the variability in the blank (baseline) and determined that the concentration of MNP on the electrode contributed to this variability. We improved the precision with which we perform IMS on the samples by building a custom magnetic rack to ensure that minimal MNP were lost during the IMS step leading to more consistent MNP between samples. We also generated samples of MNP directly bound to AuNP wherein the number of MNP remained constant while the number of bound AuNPs was varied across several magnitudes. Using this sample to calibrate the AuNP limit of detection we predicted an AuNP LOD of approximately 10⁷ nanoparticles in the presence of 20 ug of MNP. This result is shown in FIG. 20C. Note that the blank control has a baseline of 150 nA while the sample labeled D3 contains an equal number of MNP as the blank, but with the addition of approximately 10⁷ AuNP immobilized to the MNP.

The current best limit of detection for Zikavirus in a 500 μL sample was shown to be 20 ng/mL with the potential for 1 ng/mL detection within reach. These data are shown in FIG. 20D.

The crux of previous electrochemical detection methods we have employed has been the reliability and limit of detection of disposable, screen-printed carbon electrode. Additionally, antibody efficiency, nonspecific binding, and electrochemical signal inhibitors have also been areas of difficulty. The Zensens SPCE have proven to be more robust, reliable, and sensitive than Dropsens SPCE. Magnetic capture and precision in IMS have contributed to reducing the variation in baseline signals.

This result is of significant importance because it shows that we are now able to employ the same detection techniques proven successful in bacteria and parasites but now in virus samples. Such embodiments can be beneficially aimed at using a single platform technology to detect a gambit of pathogens, including reliable detection of 1 ng/mL Zika virus.

### EXAMPLE 25

### Norovirus Detection Using Screen Printed Carbon Electrodes

### Objective

Evaluate the limit of detection when aptamers are used in place of antibody.

### Method

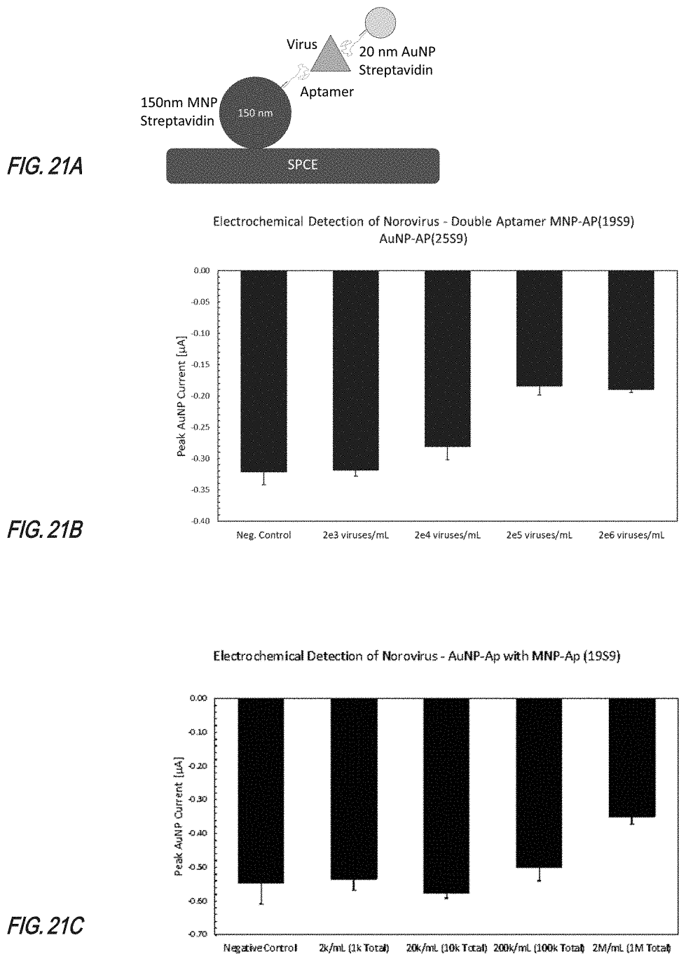

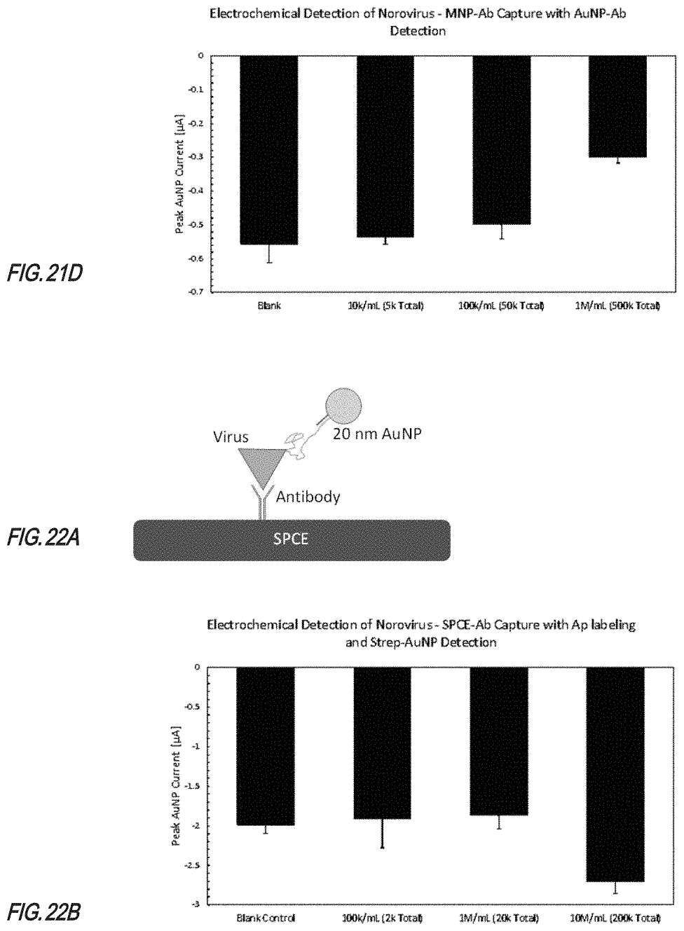

In a similar protocol described above, streptavidin conjugated magnetic nanoparticles (MNP) and gold nanoparticles (AuNP) were reacted with biotin labeled aptamers (Ap) (NVII-1959 AuNP and NVII-1959 MNP) using standard protocols. A serial dilution of Norovirus in PBS was used to prepare experiment samples. MNP-Ap were incubated with each sample for 30 minutes on a tube rotator (32 rpm end-over-end). Then, AuNP-Ap were added to the samples and incubated for an additional 30 minutes on a tube rotator (32 rpm end-over-end). The samples were washed in PBS using immunomagnetic separation to isolate the sandwich complexes from unbound AuNP and background elements. The samples were resuspended in 50 μL of PBS, mixed with 50 μL of 0.2M HCl, and added to the electrode. A magnet was then used to concentrate the MNP on the working electrode surface and square wave voltammetry was immediately performed to generate an electrochemical signal. The model in FIG. 21A describes the theoretical schematic of the detection assay.

### Results and Discussion

The rationale for conducting this experiment was to evaluate and potentially resolve the nonspecific binding observed in nanoparticles conjugated to antibody by replacing the antibody with aptamer. Aptamers generated by the SELEX process are inherently designed to limit nonspecific binding. The results of the experiment are shown in FIG. 21B.

We have observed an unanticipated but consistent result in this and similar tests where increasing concentrations of the virus correlate with lower electrochemical signal. Based on these data, the limit of detection is approximately 20k viruses per mL.

Previous results indicated that 2e4 virus per mL were possible with this method. In this modified variant, we wanted to determine if an identical aptamer on both MNP and AuNP would result in increased LOD. The results of the experiment are shown in FIG. 21C.

The predicted trend, increasing concentrations of the virus correlate with a lower electrochemical signal, was again observed. However, the LOD was not as low as in the experiment, where different aptamers were used on the AuNP and MNP. Based on these data, the limit of detection is approximately 100k viruses per mL. While other methods have shown lower LOD, this technique remains relevant because the same technology has been shown effective in bacteria and parasite detection. In order to be useful in a real-world virus detection scenario, a preconcentration step could be beneficial and thereby enable a multi-pathogen-capable detection platform that can process high volume samples.

### EXAMPLE 26

### Norovirus Detection Using Screen-Printed Carbon Electrodes—SPCE Capture

### Objective

Evaluate the limit of detection when SPCE immobilized antibody is used to capture viruses followed by incubation with biotinylated aptamer then streptavidin-conjugated AuNP.

### Method

Anti-Norovirus antibodies were passively adsorbed to SPCE, as described herein and as illustrated in FIG. 22A. A serial dilution of Norovirus in PBS was used to prepare experiment samples. The biotinylated aptamer was synthesized, and 20 nm AuNP-streptavadin was obtained from Nanopartz.

### Procedure for Passive Adsorption of Antibody to SPCE

Custom parafilm working electrode isolators (4 mm hole diameter) were placed on each electrode. Antibody was added to the working electrode (100 μg/mL in PBS) and incubated overnight at 4° C. Following incubation was a rinse step with 1 mL PBS and blocking with 10% BSA for 1 h (0.1 g BSA in 1 mL PBS). The blocking solution was removed and the a wash step performed with 1 mL PBS before use.

### Procedure for Capture Virus on SPCE

20 μL of virus sample was added to the electrode and incubated for 30 minutes. A rinse step was performed with 1 mL PBS, and 20 μL of prepared aptamer (NVII-1959 10 μM) was added and incubated for 15 minutes followed by addition of 20 μL of AuNP Streptavidin 20 nm and incubation for 15 minutes. Another rinse step was performed with 1 mL PBS before proceeding to the Echem protocol, such as that disclosed herein.

### Results and Discussion

As shown in the plot illustrated in FIG. 22B, the negative control is stable. We again observe a previously seen trend where lower concentrations of virus display increasing signal amplitude in samples without MNP. This was observed in the PCR validated virus recovery of rotavirus using MNP and again here with Norovirus. Based on the data collected in this experiment, the detection of 100k viruses in a 20 μL sample is probable.

While multiple experiments and their results were presented herein, the results of these experiments are dependent on the parameters and the conditions under which these experiments were conducted. While certain theories for these results of these experiments may be expressed herein, this disclosure is not bound by any particular theory.

While the examples above have been described with specific materials, purchased devices, and various parameters for each of these experiments, the principles contained herein may include variations from the specific: materials, purchased devices, and various parameters included in these experiments. Any appropriate materials, test equipment, or other types of parameters may be used to carry out the principles disclosed herein.

What is claimed is:

1. A method for detecting a pathogen in a sample, comprising:
introducing a nonmagnetic metal into the sample, the nonmagnetic metal coupled to an anti-pathogen antibody that is specific for the pathogen and configured to form a complex of the nonmagnetic metal and the pathogen;
removing free nonmagnetic metal from the sample, wherein the free nonmagnetic metal is not forming the complex;
detecting a presence of the nonmagnetic metal in the sample; and
determining a concentration of the pathogen in the sample based on the presence of the nonmagnetic metal.

2. The method of claim 1, wherein the nonmagnetic metal includes at least one non-ferrous metal nanoshell.

3. The method of claim 2, wherein the nonmagnetic metal includes gold.

4. The method of claim 1, further comprising collecting the complex of the nonmagnetic metal and the pathogen on a surface of an electrode.

5. The method of claim 4, wherein the complex further includes magnetic objects that are complexed with the anti-pathogen antibody.

6. The method of claim 5, wherein detecting the presence of the nonmagnetic metal in the sample includes performing voltammetry on the electrode.

7. The method of claim 6, wherein detecting the presence of the nonmagnetic metal in the sample includes comparing a signal from the voltammetry to signals from other samples known to contain the pathogen.

8. The method of claim 5, further including introducing the magnetic objects into the sample prior to introducing the nonmagnetic metal.

9. The method of claim 8, further comprising magnetically separating immunocaptured complex from a remaining portion of the sample, the immunocaptured complex including the magnetic objects, the nonmagnetic metal, the pathogen, and the anti-pathogen antibody.

10. The method of claim 8, further comprising holding the magnetic objects in place within a tube with a magnet while removing non-target debris from the tube.

11. The method of claim 1, further comprising depositing a portion of the nonmagnetic metal bound to the pathogen on an electrode through an aqueous solution and drying the electrode with the deposited nonmagnetic metal bound to the pathogen.

12. The method of claim 11, wherein determining the concentration of the pathogen in the sample based on the presence of the nonmagnetic metal comprises:
placing a solution containing charge carriers on the electrode;
passing an electrical signal through the electrode;
measuring a resulting electrical characteristic of the electrical signal; and
comparing the resulting electrical characteristic to a database of electrical characteristics of known pathogens concentrations to determine the presence of a pathogen in the sample.

13. A system for detecting at least one pathogen in a sample, comprising:
a first antibody coupled to a nonmagnetic metal;
a second antibody coupled to a magnetic object, wherein the first and second antibodies are configured to form a complex comprising a pathogen, the nonmagnetic metal and the magnetic object;
a sample mixing chamber for receiving the sample;
a magnet selectively positionable to be adjacent the sample mixing chamber;
an electrode in selective communication with the sample mixing chamber, the nonmagnetic metal configured to associate with the electrode; and
a voltmeter in electrical communication with the electrode.

14. The system of claim 13, wherein the first antibody and the second antibody are each specific for the pathogen.

15. The system of claim 13, wherein the first antibody is specific for the pathogen and the second antibody is specific to the F_{c} region of the first antibody.

16. A method for detecting a pathogen in a sample, comprising:
binding a pathogen to a magnetic object by introducing the magnetic object into the sample, wherein the magnetic object is complexed with a first antibody that is specific to the pathogen to form a first complex;
forming a second complex including the first complex and a nonmagnetic metal by introducing the nonmagnetic metal into the sample with the first complex, wherein the nonmagnetic metal is complexed with a second antibody specific to the pathogen;
retaining the second complex in the sample with a magnet;
removing a portion of the nonmagnetic metal not incorporated into the second complex; and
detecting the presence of the nonmagnetic metal in the sample, wherein the presence of the nonmagnetic material indicates the presence of the pathogen.

17. The method of claim 16, wherein the specificity of the first antibody and the second antibody are to the same epitopes of the pathogen.

18. The method of claim 16, further comprising depositing the second complex on an electrode through an aqueous solution.

19. The method of claim 18, further comprising determining a concentration of the pathogen in the sample based on the presence of the nonmagnetic metal.

20. The method of claim 19, wherein determining the concentration of the pathogen in the sample based on the presence of the nonmagnetic metal comprises:
passing an electrical signal through the electrode;
measuring a resulting electrical characteristic of the electrical signal; and
comparing the resulting electrical characteristic to a database of electrical signals of known pathogens concentrations to determine the concentration of the pathogen in the sample.
